(19) United States
(12) Patent Application Publication
(10) Pub. No.: US 2007/0249691 A1
(43) Pub. Date: Oct. 25, 2007

(54) Mercaptoimidazoles as Ccr2 Receptor Antagonists

(76) Inventors: Gustaaf Maria Boeckx, Oud-Turnhout (BE); Guy Rosalia Eugeen Van Lommen, Berlaar (BE); Julien Georges Pierre-Olivier Doyon, Turnhout (BE); Erwin Coesemans, Nijlen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/569,268

(22) PCT Filed: May 24, 2005
(86) PCT No.: PCT/EP05/52369

§ 371 Date: Nov. 17, 2006

(30) Foreign Application Priority Data
May 26, 2004 (EP) PCT EP04 050933

(51) Int. Cl. A61K 31/4164; A61K 31/421; A61K 31/4245; A61K 31/426; A61P 29/00; C07D 233/84; C07D 261/06; C07D 271/10; C07D 277/18

(52) U.S. Cl. 514/364; 514/370; 514/378; 514/398; 548/143; 548/199; 548/247; 548/324.1

(57) ABSTRACT

The present invention relates to a compound of formula (I) 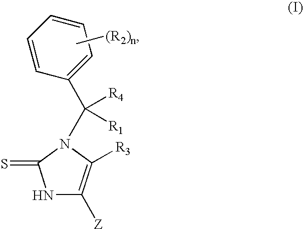
a N-oxide, a pharmaceutically acceptable addition salt, a quaternary amine, a polymorphic form or a stereochemically isomeric form thereof, wherein R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl; each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy; R₃ represents hydrogen, cyano, optionally substituted C₁₋₆alkyl, C(═O)—O—R₅, C(═O)—NR₆ₐR_{6b}, C(═S)—NR₆ₐR_{6b}, S(═O)₂—NR₆ₐR_{6b} or C(═O)—R₇; R₄ represents hydrogen or C₁₋₆alkyl; n is 1, 2, 3, 4 or 5; Z represents a cyclic ring system. The invention also relates to processes for preparing the compounds of formula (I), their use as CCR2 antagonists and pharmaceutical compositions comprising them.

The present invention concerns mercaptoimidazole derivatives having CCR2 receptor antagonistic properties. The invention further relates to methods for their preparation and pharmaceutical compositions comprising them. The invention also relates to the use of said compounds for the manufacture of a medicament for the prevention or the treatment of diseases mediated through activation of the CCR2 receptor, in particular the CCR2B receptor.

WO 02/066458 describes 2-thio-substituted imidazole derivatives having immunomodulating and/or inhibiting activity on the release of cytokines, especially TNF-α and IL-β.

FR 1,487,326 relates to thio-imidazole derivatives useful as analgetic and for its vasodilatation activity.

FR 6,751 M describes thio-imidazole derivatives as sedatives and analgesics.

U.S. Pat. No. 3,850,944 describes 2-mercapto-5-(3-pyridyl)-imidazole derivatives having antiinflammatory activity.

Bull. Soc. Chim. Belg., 73, pp 181-188 (1964) describes the synthesis and properties of α-arylalkylamines.

Archiv der Pharmazie, 305 (12), pp 891-901 (1972) describes the synthesis and pharmacology of N-chloracyl-benzylamines.

Annales pharmaceutiques francaises, 29(1), pp 63-70 (1971) describes the synthesis of imidazole derivatives.

The compounds of the invention differ from the prior art compounds in structure, in their pharmacological activity and/or pharmacological potency.

One aspect of the present invention relates to a compound of formula 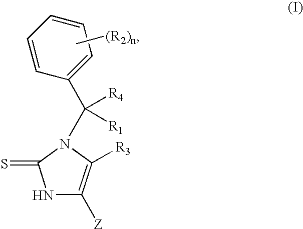
a N-oxide, a pharmaceutically acceptable addition salt, a quaternary amine, a polymorphic form or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₆₋₄alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or ²C₁₋₆alkyloxy, C(═O)—O—R₅, C(═O)—NR_{6b}, C(═S)—NR₆ₐR_{6b}, S(═O)₂—NR₆ₐR_{6b} or C(═O))—R₇;
R₄ represents hydrogen or C₁₋₆alkyl;
R₅ represents hydrogen, C₁₋₆alkyl, hydroxyC₁₋₆alkyl, C₂₋₆alkenyl C₂₋₆alkynyl polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl mono- or di(C₁₋₄alkyl)aminocarbonylC₁₋₆alkyl or aryl;
R₆ₐ and R_{6b} each independently represent hydrogen, C₁₋₆alkyl amino, mono- or di(C₁₋₄alkyl)amino, arylNH—, aminoC₁₋₆alkyl, mono- or di(C₁₋₄alkyl)amino-C₁₋₆alkyl C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 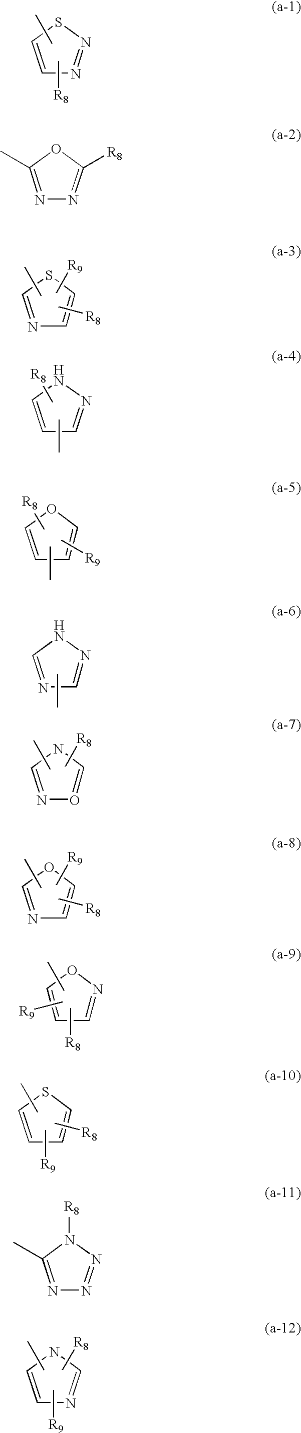 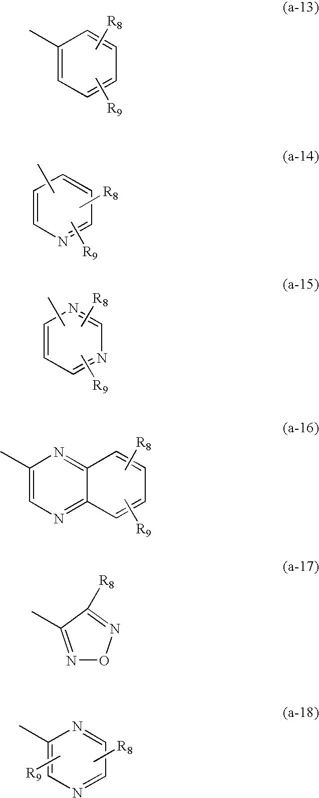
   each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
   each R₉ independently represents hydrogen, halo or C₁₋₆alkyl;
n is 1, 2, 3, 4 or S;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro;
   provided that
1-(3,4-dimethoxybenzyl)-4-phenyl-1H-imidazole-2-thiol; and
1-(o-chlorobenzyl)-5-ethyl-4-phenyl-imidazole-2-thiol
   are not included.

The present invention also relates to the use of a compound for the manufacture of a medicament for preventing or treating diseases mediated through activation of the CCR2 receptor, in particular for preventing or treating inflammatory diseases, wherein said compound is a compound of formula (I) 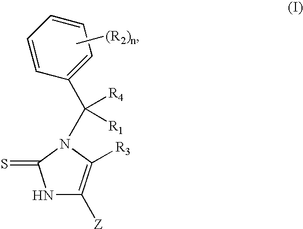
a N-oxide, a pharmaceutically acceptable addition salt, a quaternary amine, a polymorphic form or a stereochemically isomeric form thereof, wherein
R₁ represents hydrogen, C₁₋₄alkyl, C₃₋₇cycloalkyl, C₁₋₆alkyloxyC₁₋₄alkyl, di(C₁₋₆alkyl)aminoC₁₋₆alkyl, aryl or heteroaryl;
each R₂ independently represents halo, C₁₋₆alkyl, C₁₋₄alkyloxy, C₁₋₆alkylthio, polyhaloC₁₋₆alkyl polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆-alkyl aminocarbonylC₁₋₆alkyl mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₆-alkyl, mono- or di(C₁₋₄alkyl)amino C₁₋₆alkyl C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl C₂₋₆alkenyl, C₂₋₆alkynyl, polyhaloC₁₋₆alkyl, C₁₋₆alkyloxyC₁₋₆alkyl, aminoC₁₋₆alkyl mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 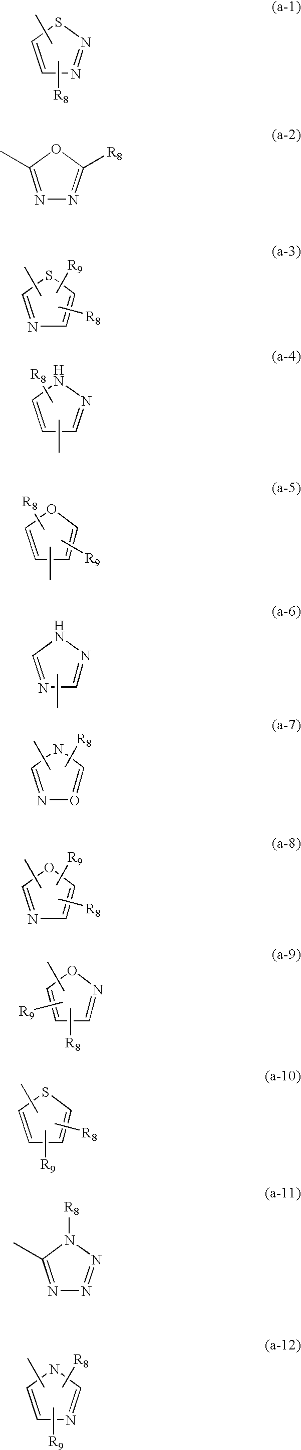 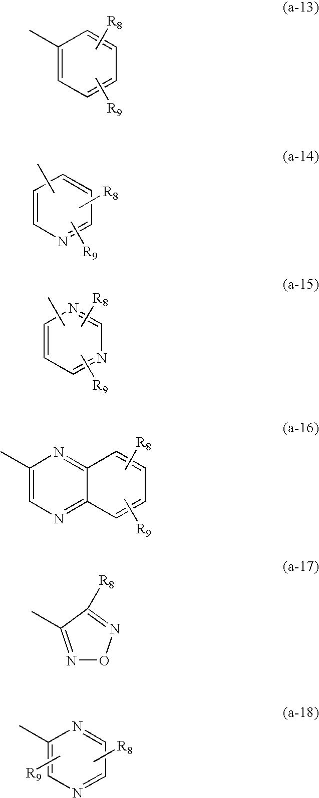
   each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
   each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

More in particular, the present invention also relates to the use of a compound for the manufacture of a medicament for preventing or treating diseases mediated through activation of the CCR2 receptor, in particular for preventing or treating inflammatory diseases, wherein said compound is a compound of formula (I) 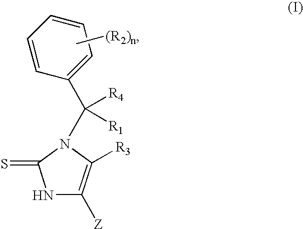
a N-oxide, a pharmaceutically acceptable addition salt, a quaternary amine, a polymorphic form or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄-alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₂₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₄alkyl, mono- or di(C₁₋₄alkyl)amino C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 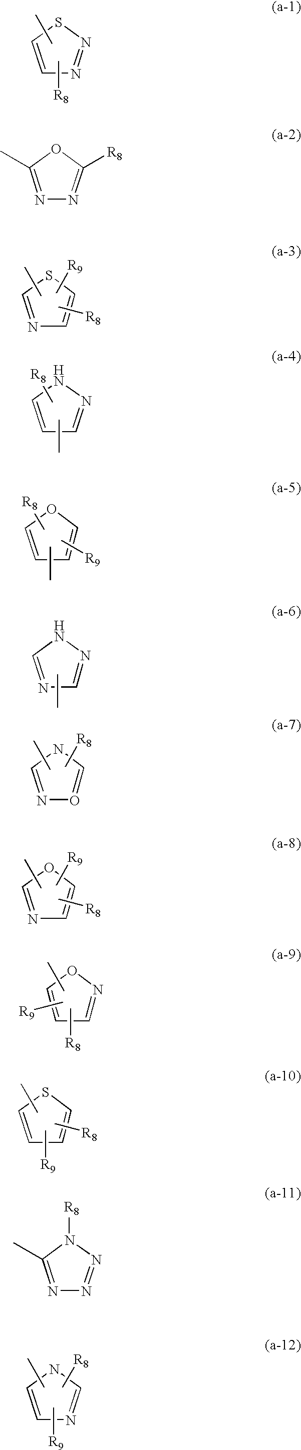 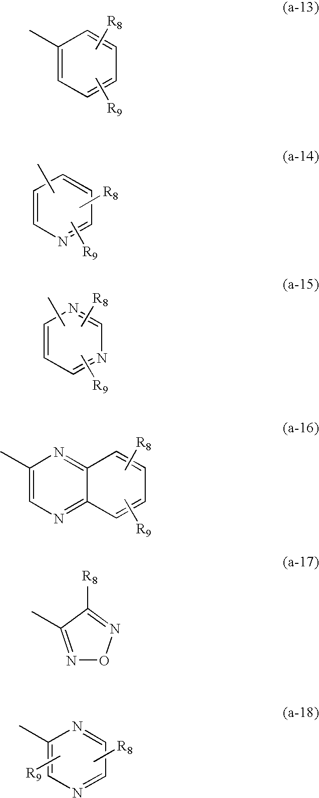
   each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
   each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro;
   provided that
1-(3,4-dimethoxybenzyl)-4-phenyl-1H-imidazole-2-thiol; and
1-(o-chlorobenzyl)-5-ethyl-4-phenyl-imidazole-2-thiol
   are not included.

A particular embodiment of the present invention are those compounds of formula (I) as defined hereinabove or hereinafter provided that 1-(3,4-dimethoxybenzyl)-4-phenyl-1H-imidazole-2-thiol; 1-(o-chlorobenzyl)-5-ethyl-4-phenyl-imidazole-2-thiol or pharmaceutically acceptable addition salts thereof are not included.

As used hereinbefore or hereinafter C₁₋₄alkyl as a group or part of a group defines straight or branched chain saturated hydrocarbon radicals having from 1 to 4 carbon atoms such as methyl, ethyl, propyl, 1-methylethyl, butyl; C₁₋₆alkyl as a group or part of a group defines straight or branched chain saturated hydrocarbon radicals having from 1 to 6 carbon atoms such as the group defined for C₁₋₆alkyl and pentyl, hexyl, 2-methylbutyl and the like; C₃₋₇cycloalkyl is generic to cyclopropyl, cyclobutyl, cyclopentyl, cyclohexyl and cycloheptyl; C₂₋₆alkenyl defines straight and branched chain hydrocarbon radicals having from 2 to 6 carbon atoms containing a double bond such as ethenyl, propenyl, butenyl, pentenyl, hexenyl and the like; C₂₋₆alkynyl defines straight and branched chain hydrocarbon radicals having from 2 to 6 carbon atoms containing a triple bond such as ethynyl, propynyl, butynyl, pentynyl, hexynyl and the like.

As used hereinbefore, the term (═O) forms a carbonyl moiety when attached to a carbon atom, a sulfoxide moiety when attached to a sulfur atom and a sulfonyl moiety when two of said terms are attached to a sulfur atom.

The term halo is generic to fluoro, chloro, bromo and iodo. As used in the foregoing or hereinafter, polyhalomethyl as a group or part of a group is defined as mono- or polyhalosubstituted methyl, in particular methyl with one or more fluoro atoms, for example, difluoromethyl or trifluoromethyl; polyhaloC₁₋₆alkyl as a group or part of a group is defined as mono- or polyhalosubstituted C₁₋₆alkyl, for example, the groups defined in polyhalomethyl, 1,1-difluoro-ethyl and the like. In case more than one halogen atoms are attached to an alkyl group within the definition of polyhalomethyl or polyhaloC₁₋₆alkyl, they may be the same or different.

The term heteroaryl in the definition of R₁ or R₇ is meant to include all the possible isomeric forms of the heterocycles, for instance, pyrrolyl comprises 1H-pyrrolyl and 2H-pyrrolyl.

The aryl, heteroaryl, heterocyclic ring systems or cyclic ring systems listed in the definitions of the substituents of the compounds of formula (I) (see for instance R₁, R₅, R₇ and Z) as mentioned hereinabove or hereinafter may be attached to the remainder of the molecule of formula (I) through any ring carbon or heteroatom as appropriate, if not otherwise specified. Thus, for example, when heteroaryl is imidazolyl, it may be 1-imidazolyl, 2-imidazolyl, 4-imidazolyl and the like.

When any variable (eg. R₆ₐ, R_{6b}) occurs more than one time in any constituent, each definition is independent.

Lines drawn from substituents into ring systems indicate that the bond may be attached to any of the suitable ring atoms. When the lines are drawn into bicyclic ring systems, it indicates that the bond may be attached to any of the suitable ring atoms of any one of the two cycles of the bicyclic ring system.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable addition salts as mentioned hereinabove are meant to comprise the therapeutically active non-toxic acid addition salt forms which the compounds of formula (I) are able to form. The latter can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of formula (I) containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanediol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of formula (I) are able to form. Examples of such forms are e.g. hydrates, alcoholates and the like.

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several tertiary nitrogen atoms are oxidized to the so-called N-oxide.

It will be appreciated that some of the compounds of formula (I) and their N-oxides, addition salts, quaternary amines, polymorphic forms or stereochemically isomeric forms may contain one or more centers of chirality and exist as stereochemically isomeric forms.

The term “stereochemically isomeric forms” as used hereinbefore or hereinafter defines all the possible stereoisomeric forms which the compounds of formula (I), and their N-oxides, addition salts, quaternary amines, polymorphic forms or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of formula (I) and their N-oxides, salts, solvates, quaternary amines or polymorphic forms substantially free, i.e. associated with less than 10%, preferably less than 5%, in particular less than 2% and most preferably less than 1% of the other isomers. Thus, when a compound of formula (I) is for instance specified as (E), this means that the compound is substantially free of the (Z) isomer.

In particular, stereogenic centers may have the R- or S-configuration; substituents on bivalent cyclic (partially) saturated radicals may have either the cis- or trans-configuration. Compounds encompassing double bonds can have an E (entgegen) or Z (zusammen)-stereochemistry at said double bond. The terms cis, trans, R, S, E and Z are well known to a person skilled in the art.

Stereochemically isomeric forms of the compounds of formula (I) are obviously intended to be embraced within the scope of this invention.

Polymorphic forms of the present compounds also fall within the ambit of the present invention. The term “polymorphic forms” as used hereinbefore or hereinafter defines all possible crystalline arrangement of a particular compound. A polymorphic form of a compound is the same chemical entity, but in a different crystalline arrangement. The term “polymorphic form” is well-known to a person skilled in the art.

Polymorphic forms of pharmaceutical compounds may be of interest to those involved in the development of a suitable dosage form because if the polymorphic form is not held constant during clinical and stability studies, the exact dosage used or measured may not be comparable from one lot to the next. Once a pharmaceutical compound is produced for use, it is important to recognize the polymorphic form delivered in each dosage form to assure that the production process use the same form and that the same amount of drug is included in each dosage. Therefore, it is imperative to assure that either a single polymorphic form or some known combination of polymorphic forms is present. In addition, certain polymorphic forms may exhibit enhanced thermodynamic stability and may be more suitable than other polymorphic forms for inclusion in pharmaceutical formulations.

Some of the compounds of formula (I) may also exist in their tautomeric form. Such forms although not explicitly indicated in the above formula (I) are intended to be included within the scope of the present invention. For instance, it is intended that formula (I) includes the tautomeric form of 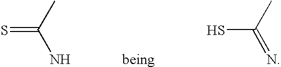
Thus, the compounds of the present invention include compounds of formula

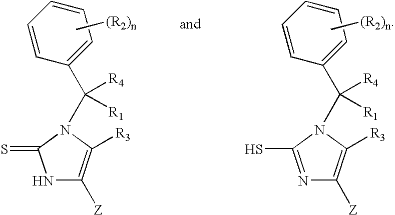

Whenever used hereinafter, the term “compounds of formula (I)” is meant to also include their N-oxide forms, their addition salts, their quaternary amines, their polymorphic forms or their stereochemically isomeric forms. Of special interest are those compounds of formula (I) which are stereochemically pure.

Whenever used hereinbefore or hereinafter that substituents can be selected each independently out of a list of numerous definitions, such as for example for R₆ₐ or R_{6b}, all possible combinations are intended which are chemically possible.

A first interesting embodiment of the present invention relates to a compound of formula 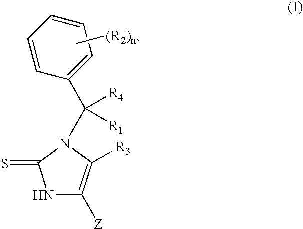
a N-oxide, a pharmaceutically acceptable addition salt, a quaternary amine or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)aminoC₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 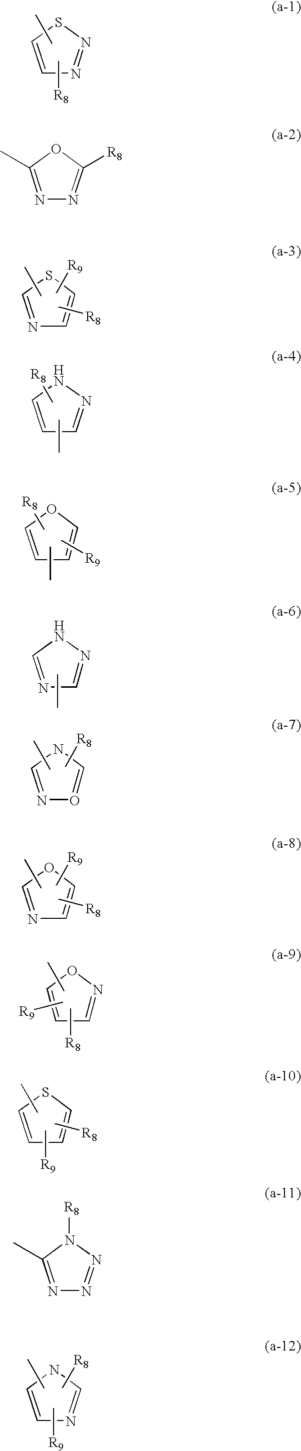 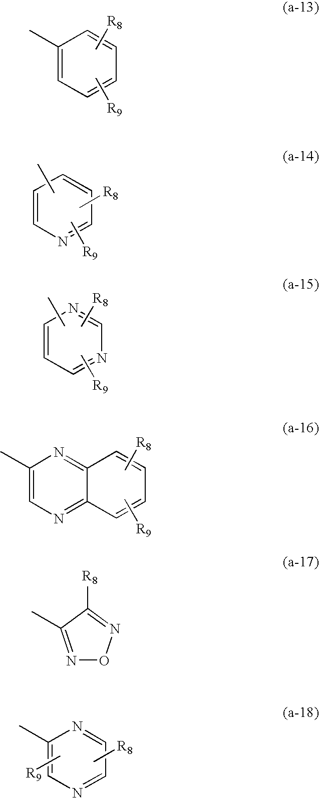
   each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
   each R₉ independently represents hydrogen, halo or C₁₋₆alkyl;
n is 1, 2, 3, 4 or S;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro;
   provided that
1-(3,4-dimethoxybenzyl)-4-phenyl-1H-imidazole-2-thiol; and
1-(o-chlorobenzyl)-5-ethyl-4-phenyl-imidazole-2-thiol
   are not included.

A second interesting embodiment of the present invention relates to a compound of formula 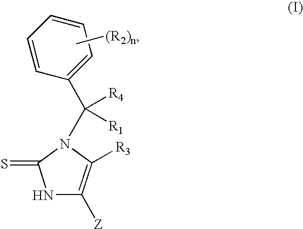
wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—N₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl polyhaloC₁₋₆alkyl C₁₋₆alkyloxyC₁₋₆alkyl, aminoC₁₋₆alkyl mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl amino, mono- or di(C₁₋₄alkyl)amino, arylNH—, aminoC₁₋₆alkyl, mono- or di(C₁₋₄alkyl)aminoC₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 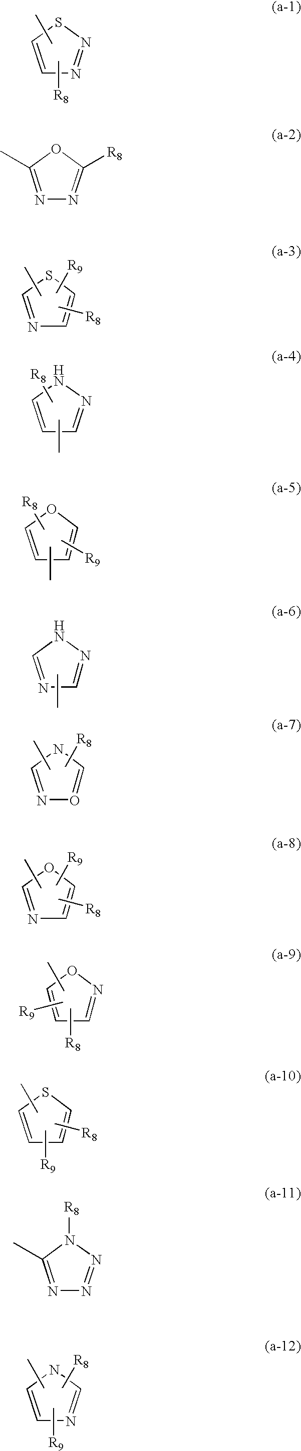 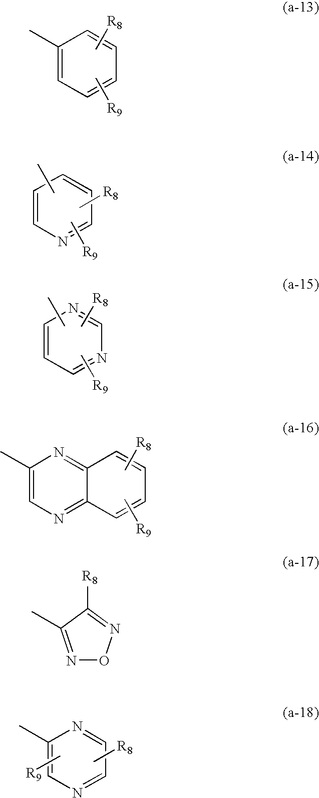
   each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₆-alkyl)amino, hydroxyC₁₋₆alkylamino, aryl, aryloxy, piperidinyl, piperidinylamino, morpholinyl, piperazinyl or nitro;
   each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro;
   provided that
1-(3,4-dimethoxybenzyl)-4-phenyl-1H-imidazole-2-thiol; and
1-(o-chlorobenzyl)-5-ethyl-4-phenyl-imidazole-2-thiol
   are not included.

A third interesting embodiment are the compounds of formula (I) or any subgroup thereof as mentioned hereinbefore as interesting embodiment wherein R₃ represents hydrogen, cyano, C(═O)—O—R₅, C(═O)—NR₆ₐR_{6b}, C(═S)—NR₆ₐR_{6b}, S(═O)₂—NR₆ₐR_{6b} or C(═O)—R₇.

A fourth interesting embodiment are the compounds of formula (I) or any subgroup thereof as mentioned hereinbefore as interesting embodiment wherein R₃ represents cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇. A fifth interesting embodiment are the compounds of formula (I) or any subgroup thereof as mentioned hereinbefore as interesting embodiment wherein R₃ represents cyano, C(═O)—O—R₅, C(—O)—NR₆ₐR_{6b} C(═S)—NR₆ₐR_{6b}, S(═O)₂—NR₆ₐR_{6b} or C(═O)—R₇.

A sixth interesting embodiment are the compounds of formula (I) or any subgroup thereof as mentioned hereinbefore as interesting embodiment wherein R₃ represents hydrogen, cyano, C(═O)—O—R₅, C(═O)—NR₆ₐR_{6b} or C(═O)—R₇; preferably C(═O)—O—R₅; more preferably C(—O)—O—C₁₋₆alkyl; most preferred C(═O)—O—CH₃.

A seventh interesting embodiment are the compounds of formula (I) or any subgroup thereof as mentioned hereinbefore as interesting embodiment wherein Z is other than 3-pyridyl.

An eight interesting embodiment are the compounds of formula (I) or any subgroup thereof as mentioned hereinbefore as interesting embodiment wherein Z is a cyclic ring system selected from (a-1), (a-2), (a-3), (a-4), (a-5), (a-6), (a-7), (a-9), (a-10), (a-11), (a-12), (a-13), (a-14), (a-15) or (a-16) as defined hereinabove; preferably a cyclic ring system selected from (a-1), (a-2), (a-3), (a-4), (a-9), (a-10), (a-12), (a-13), (a-14), (a-16) or (a-18); more preferably a cyclic ring system selected from (a-1), (a-2), (a-3), (a-9), (a-10), (a-12), (a-13), (a-14) or (a-16); even more preferably a cyclic ring system selected from (a-1), (a-2), (a-9), (a-10) or (a-13); most preferred a cyclic ring system of formula (a-9).

A ninth interesting embodiment are the compounds of formula (I) or any subgroup thereof as mentioned hereinbefore as interesting embodiment wherein Z is a cyclic ring system selected from (a-2), (a-7) or (a-9) or a cyclic ring system selected from (a-2), (a-8) or (a-10); preferably wherein Z is a cyclic ring system selected from (a-2), (a-7) or (a-9); more preferably wherein Z is a cyclic ring system selected from (a-2), (a-7) or (a-9) and wherein R₃ represents C(═O)—O—R₅.

A tenth interesting embodiment are the compounds of formula (I) or any subgroup thereof as mentioned hereinbefore as interesting embodiment wherein n is 2 or 3; preferably wherein n is 2.

An eleventh interesting embodiment are the compounds of formula (I) or any subgroup thereof as mentioned hereinbefore as interesting embodiment wherein n is 2 and said two R² substituents are placed in meta and para postion.

A twelfth interesting embodiment are the compounds of formula (I) or any subgroup thereof as mentioned hereinbefore as interesting embodiment wherein R₂ represents halo, C₁₋₆alkyl, C₁₋₄alkyloxy or polyhaloC₁₋₆alkyl; preferably halo or polyhaloC₁₋₆alky, in particular chloro, fluoro or trifluoromethyl; most preferred halo, in particular chloro or fluoro, more in particular fluoro.

A thirteenth interesting embodiment are the compounds of formula (I) or any subgroup thereof as mentioned hereinbefore as interesting embodiment wherein R₁ is hydrogen, methyl, ethyl, n-propyl, methoxymethyl, cyclohexyl, cyclopropyl, dimethylaminomethyl, 2-thienyl, 3,4-dichlorophenyl; preferably R₁ is C₁₋₆alkyl or C₁₋₆alkyloxyC₁₋₆alkyl, in particular methyl, ethyl, propyl, methoxymethyl, more in particular methyl, ethyl, n-propyl or methoxymethyl; more preferably R₁ is C₁₋₆alkyl, in particular methyl, ethyl and propyl, more in particular methyl, ethyl or n-propyl; most preferred R₁ is ethyl.

A fourteenth interesting embodiment are the compounds of formula (I) or any subgroup thereof as mentioned hereinbefore as interesting embodiment wherein R₄ is hydrogen.

A fifteenth interesting embodiment are the compounds of formula (I) or any subgroup thereof as mentioned hereinbefore as interesting embodiment which are stereochemically pure.

A sixteenth interesting embodiment are the compounds of formula (I) or any subgroup thereof as mentioned hereinbefore as interesting embodiment wherein the carbon atom carrying the R₁ and R₄ substituent has the (S) configuration, i.e. a compound of formula (I′) 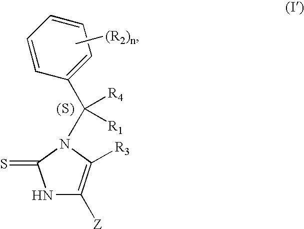
a N-oxide, a pharmaceutically acceptable addition salt, a quaternary amine, a polymorphic form or a stereochemically isomeric form thereof.

Also interesting compounds are those compounds of formula (I) wherein one or more, preferably all of the following restrictions apply:
a) R₁ represents C₁₋₆alkyl or C₁₋₆alkyloxyC₁₋₆alkyl, especially methyl, ethyl, propyl or methoxymethyl;
b) R₂ represents halo, polyhaloC₁₋₆alkyl or aryloxy, especially halo, e.g. chloro or fluoro;
c) R₃ represents hydrogen, cyano, C(═O)—O—R₅, C(═O)—NR₆ₐR_{6b} or C(═O)—R₇;
d) Z represents a ring system selected from (a-1), (a-2), (a-3), (a-4), (a-5), (a-6), (a-7), (a-9), (a-10), (a-11), (a-12), (a-13), (a-14), (a-15) or (a-16);
e) R₄ represents hydrogen;
f) n is 2 or 3.

Also interesting compounds are those compounds of formula (I) wherein one or more of the following restrictions apply:
a) R₁ represents C₁₋₆alkyl or C₁₋₆alkyloxyC₁₋₆alkyl, especially methyl, ethyl, propyl or methoxymethyl;
b) R₂ represents halo, especially chloro or fluoro;
c) R₃ represents hydrogen; cyano; C(═O)—O—R₅ wherein R₅ is preferably hydrogen, C₁₋₆alkyl or C₁₋₆alkyloxyC₁₋₆alkyl; C(═O)—NR₆ₐR_{6b} wherein R₆ₐ and R_{6b} are preferably hydrogen or hydroxyC₁₋₆alkyl; C(═O)—R₇ wherein R₇ is preferably optionally substituted thiazolyl;
d) Z represents a ring system selected from (a-1), (a-2), (a-3), (a-4), (a-9), (a-10), (a-12), (a-13), (a-14), (a-16) or (a-18).
e) R₄ represents hydrogen;
f) n is 2.

Further interesting compounds are those compounds of formula (I) wherein one or more of the following restrictions apply:
a) R₁ represents C₁₋₆alkyl, especially ethyl or propyl;
b) R₂ represents halo, especially chloro or fluoro;
c) R₃ represents C(═O)—O—R₅ or C(═O)NR₆ₐR_{6b};
d) Z represents a cyclic ring system selected from (a-1), (a-2), (a-9), (a-10) or (a-13);
e) R₄ represents hydrogen;
f) n is 2.

Yet further interesting compounds are those compounds of formula (I) wherein one or more of the following restrictions apply:
a) R₁ represents ethyl or propyl;
b) R₂ represents chloro or fluoro;
c) R₃ represents C(═O)—O—R₅, especially C(═O)—O—CH₃;
d) Z represents cyclic ring system (a-9);
e) R₄ represents hydrogen;
f) n is 2.

Preferred compounds of formula (I) are compounds 31, 6, 27, 9, 24, 40, 25, 7, 26, 45, 48, 49, 43, 36, 16, 28, 33, 32, 34, 51, 52 or 53; a N-oxide, a pharmaceutically acceptable addition salt, a quaternary amine, a polymorphic form or a stereochemically isomeric form thereof.

More preferred compounds of formula (I) are compounds 26, 48, 43, 52 or 53, especially compound 26, 43 or 53; a N-oxide, a pharmaceutically acceptable addition salt, a quaternary amine, a polymorphic form or a stereochemically isomeric form thereof.

Most preferred is any one of the following:

a compound of formula (I) wherein the compound is (S)-3-[1-(3,4-difluoro-phenyl)propyl]-5-isoxazol-5-yl-2-thioxo-2,3-dihydro-1H-imidazole-4-carboxylic acid methyl ester, a N-oxide, a pharmaceutically acceptable addition salt, a quaternary amine or a polymorphic form thereof;

a compound of formula (I) wherein the compound is (S)-3-[1-(3,4-difluoro-phenyl)propyl]-5-isoxazol-5-yl-2-thioxo-2,3-dihydro-1H-imidazole-4-carboxylic acid methyl ester or a N-oxide thereof;

a compound of formula (I) wherein the compound is (S)-3-[1-(3,4-difluoro-phenyl)propyl]-5-isoxazol-5-yl-2-thioxo-2,3-dihydro-1H-imidazole-4-carboxylic acid methyl ester or a pharmaceutically acceptable addition salt thereof;

a compound of formula (I) wherein the compound is (S)-3-[1-(3,4-difluoro-phenyl)propyl]-5-isoxazol-5-yl-2-thioxo-2,3-dihydro-1H-imidazole-4-carboxylic acid methyl ester or a quaternary amine thereof;

a compound of formula (I) wherein the compound is (S)-3-[1-(3,4-difluoro-phenyl)propyl]-5-isoxazol-5-yl-2-thioxo-2,3-dihydro-1H-imidazole-4-carboxylic acid methyl ester; or

a compound of formula (I) wherein the compound is (S)-3-[1-(3,4-difluoro-phenyl)propyl]-5-isoxazol-5-yl-2-thioxo-2,3-dihydro-1H-imidazole-4-carboxylic acid methyl ester with a melting point of 128.5° C.

In general, compounds of formula (I) wherein R₃ represents hydrogen, said compounds being represented by formula (I-a), can be prepared by reacting an intermediate of formula (II-a) or (II-b) with KSCN in the presence of a suitable acid, such as for example hydrochloric acid and the like, and a suitable solvent, such as for example an alcohol, e.g. ethanol, optionally in the presence of water.

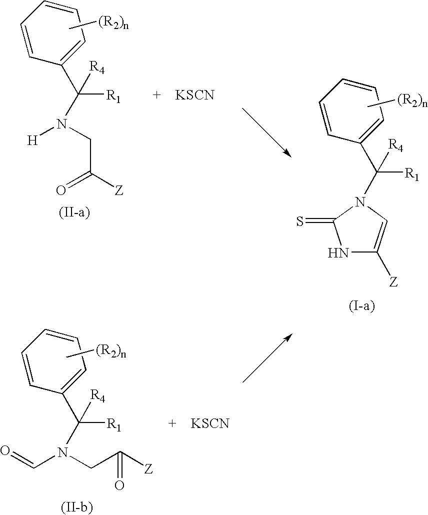

Compounds of formula (I) wherein R₃ is other than hydrogen, said R₃ being represented by R_{3′} and said compounds being represented by formula (I-b), can be prepared by reacting an intermediate of formula (III) with an intermediate of formula (IV) wherein W₁ represents a suitable leaving group, such as for example C₁₋₆alkyloxy, e.g. methoxy or t-butyloxy, or halo, e.g. chloro and the like, in the presence of KSCN, a suitable acid, such as for example hydrochloric acid and the like, a suitable solvent, such as for example tetrahydrofuran, or a mixture of tetrahydrofuran and a suitable alcohol, e.g. methanol, and a suitable base, such as for example t-BuONa, LiN(isopropyl)₂ or NH[Si(CH₃)₃].

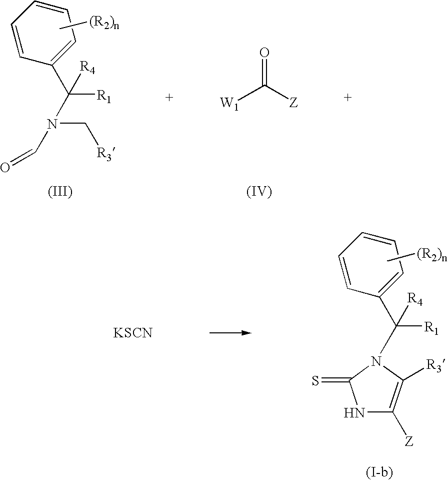

Compounds of formula (I-b) can also be prepared by reacting an intermediate of formula (V) with a suitable base, such as for example sodium hydroxide and the like, in the presence of a suitable solvent, such as for example an alcohol, e.g. ethanol.

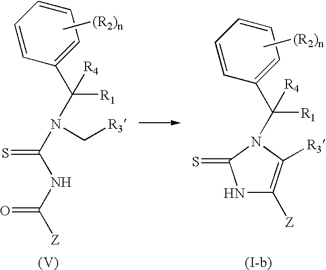

Compounds of formula (I) wherein Z represents optionally substituted 1,3,4-oxadiazole, said compounds being represented by formula (I-c), can be prepared by reacting an intermediate of formula (VI) with phosphoric trichloride or Burgess'reagent optionally in the presence of a suitable solvent, such as for example tetrahydrofuran.

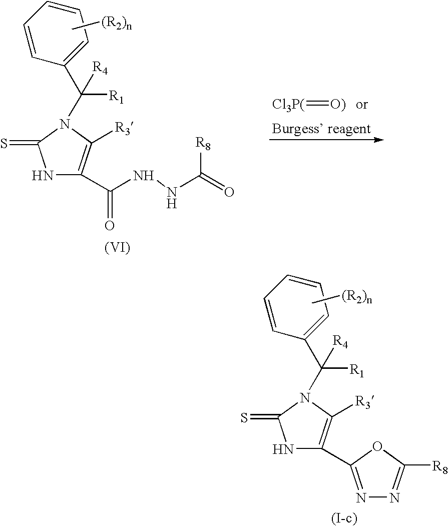

Compounds of formula (I) wherein R₃ represents C(═O)—O—R₅, wherein R₅, represents C₁₋₆alkyl or hydroxyC₁₋₆alkyl, said compounds being represented by formula (I-d), can be prepared by reacting an intermediate of formula (VII), wherein W₂ represents a suitable leaving group, such as for example halo, e.g. chloro and the like, with an appropriate alcohol of formula HO—R_{5′} in the presence of a suitable solvent, such as for example tetrahydrofuran.

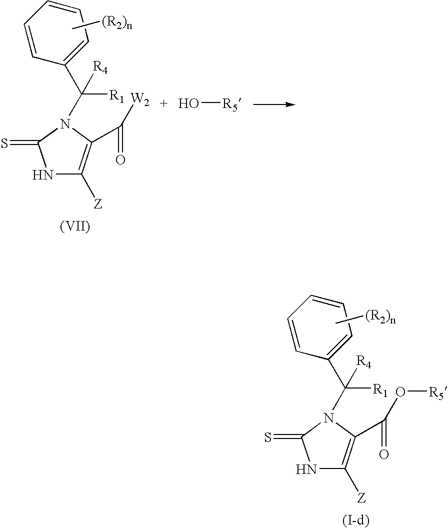

Compounds of formula (I) wherein R₃ represents C(═O)—NR₆ₐR_{6b}, said compounds being represented by formula (I-e), can be prepared by reacting an intermediate of formula (VII), with an intermediate of formula (VIII), such as for example NH₃ (or acetic acid ammonium salt), pyrrolidine and the like, in the presence of a suitable solvent, such as for example acetone, tetrahydrofuran, N,N-dimethylformamide and the like.

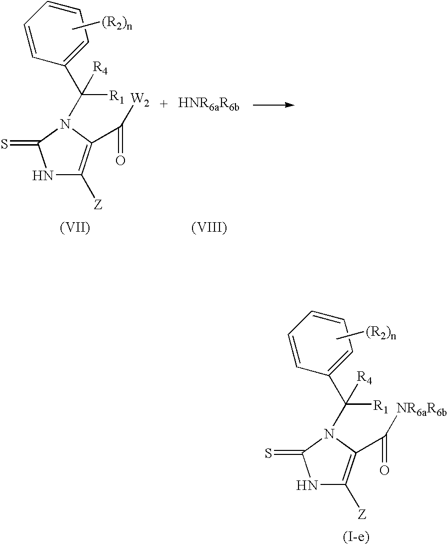

Compounds of formula (I) wherein R₃ represents CH₂—OH, said compounds being represented by formula (I-f), can be prepared by reacting an intermediate of formula (VII) with a suitable reducing agent, such as for example NaBH₄ in the presence of a suitable solvent, such as for example tetrahydrofuran.

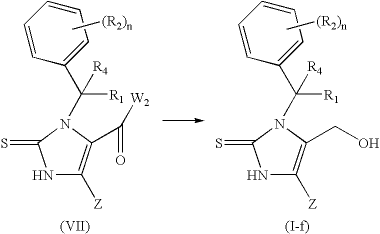

Compounds of formula (I′) can be prepared according to the above described reactions but starting from an intermediate wherein the carbon atom carrying the R₁ and R₄ substituent has the (S) configuration.

Alternatively, compounds of formula (I) wherein the carbon atom carrying the R₁ and R₄ substituent has the (R) configuration can be prepared according to the above described reactions but starting from an intermediate wherein the carbon atom carrying the R₁ and R₄ substituent has the (R) configuration.

The compounds of formula (I) may further be prepared by converting compounds of formula (I) into each other according to art-known group transformation reactions.

The compounds of formula (I) may be converted to the corresponding N-oxide forms following art-known procedures for converting a trivalent nitrogen into its N-oxide form. Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert.butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Compounds of formula (I) wherein R₃ represents C(═O)—O—C₁₋₆-alkyl, may be converted into a compound of formula (I) wherein R₃ represents CH₂—OH by reaction with a suitable reducing agent, such as for example LiHBEt₃ in the presence of a suitable solvent, such as for example tetrahydrofuran.

Compounds of formula (I) wherein R₃ represents C(═O)—O—C₁₋₆-alkyl, can also be converted into a compound of formula (I) wherein R₃ represents C(═O)—OH by reaction with a suitable base, such as NaOH, in the presence of a suitable solvent, such as for example H₂O, tetrahydrofuran or an appropriate alcohol, e.g. methanol and the like.

Compounds of formula (I) wherein R₃ represents C(═O)—O—C₁₋₆alkyl, can also be converted into a compound of formula (I) wherein R₃ represents C(═O)—NR₆ₐR_{6b}, by reaction with the appropriate base of formula NHR₆ₐR_{6b} in a suitable solvent, such as for example H₂O.

Compounds of formula (I) wherein R₃ represents C(═O)—O—H, can be converted into a compound of formula (I) wherein R₃ represents C(═O)—NR₆ₐR_{6b}, by reaction with the appropriate base of formula NHR₆ₐR_{6b} in the presence of N′-(ethylcarbonimidoyl)-N,N-dimethyl-1,3-propanediamine, 1-hydroxy-1H-benzotriazole and a suitable solvent, such as for example N,N-dimethylformamide.

Compounds of formula (I) wherein R₃ represents C(═O)—O—H, can also be converted into a compound of formula (I) wherein R₃ represents C(═O)—NH₂ by reaction with NH₄OH in the presence of SOCl₂.

Compounds of formula (I) wherein R₃ represents C(═O)—O—C₁₋₆alkyl, can also be converted into a compound of formula (I) wherein R₃ represents C(═O)—O—C₁₋₆alkyl-O—C₁₋₆alkyl, by reaction with HO—C₁₋₆alkyl-O—C₁₋₆alkyl in the presence of NaBH₄.

Compounds of formula (I) wherein R₃ represents cyano or C(═O)—O—C₁₋₆alkyl, can be converted into a compound of formula (I) wherein R₃ represents aminocarbonyl by reaction with NH₄OH.

Compounds of formula (I) wherein R₃ represents cyano, can also be converted into a compound of formula (I) wherein R₃ represents C(═S)NR₆ₐR_{6b} by reaction with hydrogen sulfide in the presence of N-ethyl-N-(1-methylethyl)-2-propanamine in a suitable solvent such as pyridine.

Compounds of formula (I) wherein R₃ represents C(═O)—NR₆ₐR_{6b} can be converted into a compound of formula (I) wherein R₃ represents C(═O)—C₁₋₆alkyl by reaction with chloroC₁₋₆alkyMg in a suitable solvent such as tetrahydrofuran.

Compounds of formula (I) wherein R₃ represents C(═O)—C₁₋₆alkyl can be converted into compounds of formula (I) wherein R₃ represents hydroxyC₁₋₆alkyl by reaction with a suitable reducing agent such as NaBH₄, in the presence of a suitable solvent such as methanol.

Some of the compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials are known compounds and may be commercially available or may be prepared according to art-known procedures.

Intermediates of formula (II-a) may be prepared by reacting an intermediate of formula (VIII) wherein W₃ represents a suitable leaving group, such as for example halo, e.g. bromo, with an intermediate of formula (IX) in the presence of a suitable base, such as for example N,N-diisopropylethanamine, and a suitable solvent, such as for example tetrahydrofuran.

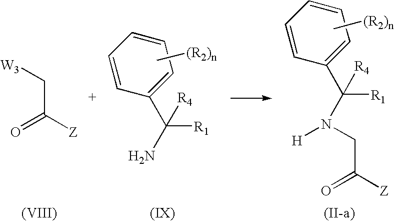

Intermediates of formula (IX) wherein R₄ represents hydrogen, said intermediates being represented by formula (IX-a), may be prepared by reacting an intermediate of formula (X) with a suitable reducing agent, such as for example H₂, in the presence of a suitable catalyst, such as for example Raney Nickel, a suitable catalyst poison, such as for example a thiophene solution, and a suitable base, such as for example NH₃.

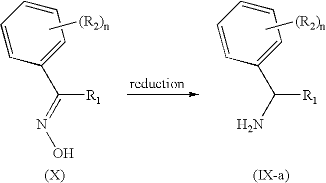

Intermediates of formula (X) may be prepared by reacting an intermediate of formula (XI) with HO—NH₂ in the presence of a suitable base, such as for example Na₂CO₃, and a suitable solvent, such as for example an alcohol, e.g. ethanol, and water.

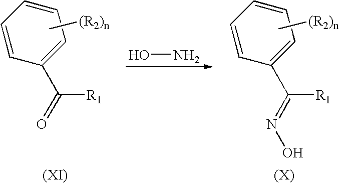

Intermediates of formula (II-b) can be prepared by reacting an intermediate of formula (VIII) with an intermediate of formula (XII) in the presence of a suitable solvent, such as for example acetonitrile.

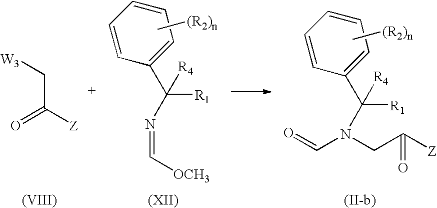

Intermediates of formula (XII) can be prepared by reacting an intermediate of formula (XIII) with trimethyloxonium and tetrafluoroborate in the presence of a suitable solvent, such as for example methylenechloride.

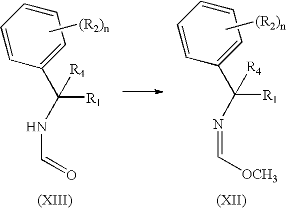

Intermediates of formula (XIII) wherein R₄ is hydrogen, said intermediates being represented by formula (XIII-a), can be prepared by reacting an intermediate of formula (XIV) with HC(—O)—NH₂ in the presence of a suitable acid, such as for example formic acid.

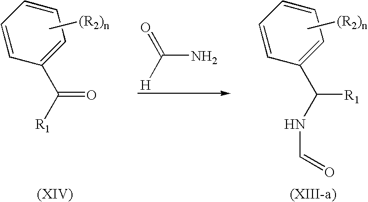

Intermediates of formula (III) can be prepared from an intermediate of formula (XV) in the presence of formic acid or a formate, such as for example n-butylformate, and in the presence of a suitable solvent, such as for example xylene.

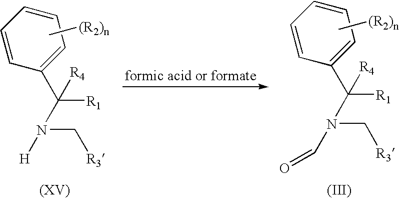

Intermediates of formula (XV) can be prepared by reacting an intermediate of formula (IX) with an intermediate of formula (XVI) wherein W₄ represents a suitable leaving group, such as for example halo, e.g. chloro and the like, in the presence of a suitable base, such as for example N,N-diethylethanamine, and a suitable solvent, such as for example N,N-dimethylformamide or tetrahydrofuran. The intermediates of formula (IX) may contain a chiral center at the carbon atom carrying the R₁ and R₄ substituent depending on the substituents representing R₁ and R₄. Said stereospecific intermediates of formula (IX) are represented by formula (IX-b). When the reaction is performed starting from a stereospecific intermediate of formula (IX-b), a stereospecific intermediate of formula (XV) is obtained, said intermediate being represented by formula (XV-a). 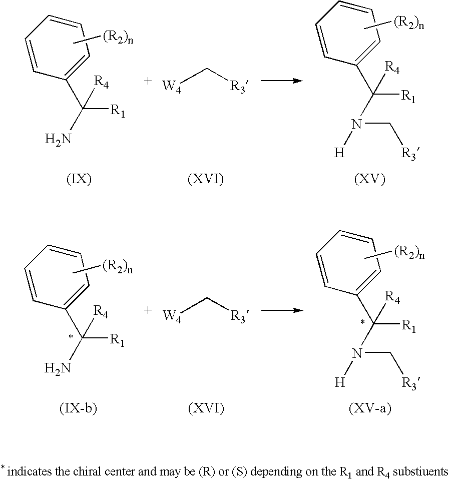
Stereospecific intermediates of formula (XV-a) can also be prepared by reacting an intermediate of formula (XV) with a suitable stereospecific resolution agent, such as for example [S—(R*,R*)]-2,3-bis[(4-methylbenzoyl)oxy]-butanedioic acid, in the presence of a suitable solvent, such as for example an alcohol, e.g. 2-propanol.

When a stereospecific intermediate of formula (XV-a) is reacted further according to the methods described hereinabove, the resulting intermediates are also stereospecific and finally the resulting final compounds are also stereospecific.

Intermediates of formula (XV) wherein R⁴ represents hydrogen, said intermediates being represented by formula (XV-b), can also be prepared by reacting an intermediate of formula (XXVI) with an intermediate of formula (XXVII) in the presence of a suitable reducing agent, such as for example H₂, a suitable catalyst, such as for example Pd on charcoal, a suitable catalyst poison, such as for example a thiophene solution, a suitable weak base, such as for example KF or potassium acetate, a suitable acid, such as for example hydrochloric acid, and a suitable solvent, such as for example an alcohol, e.g. methanol.

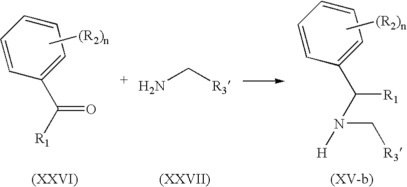

Intermediates of formula (IX-b), can be prepared by reacting an intermediate of formula (XVII) with triphenylphosphine, in the presence of a suitable solvent, such as for example tetrahydrofuran and water or by reacting an intermediate of formula (XVII) with a suitable reducing agent, such as for example H₂, in the presence of a suitable catalyst, such as for example Pt on charcoal or Pd on charcoal, and a suitable solvent, such as for example an alcohol, e.g. methanol.

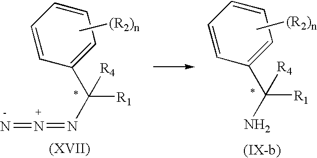

When a stereospecific intermediate of formula (IX-b) is reacted further according to the methods described hereinabove, the resulting intermediates are also stereospecific and finally the resulting final compounds are also stereospecific.

Intermediates of formula (XVII) can be prepared by reacting an intermediate of formula (XVIII) with diphenylphosphoryl azide in the presence of 2,3,4,6,7,8,9,10-octahydro-pyrimido[1,2-a]azepine and in the presence of a suitable solvent, such as for example toluene. 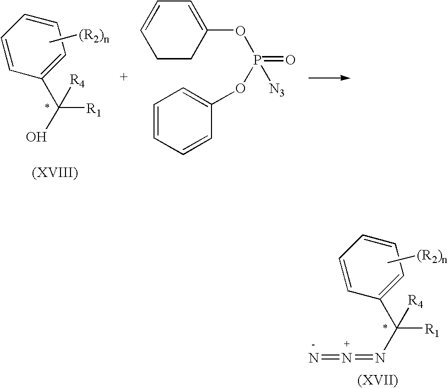
Stereospecific intermediates of formula (XVII) wherein R₄ is hydrogen and R₁ is methyl, ethyl, or n-propyl, said R₁ being represented by Alk and said intermediates being represented by formula (XVIII-a) and (XVI-b), can be prepared by reacting an intermediate of formula (XIX) with ZnAlk₂ wherein Alk represents methyl, ethyl or n-propyl, in the presence of a stereospecific catalyst, such as for example N,N′-(1R,2R)-1,2-cyclohexanediylbis[1,1,1-trifluoro]-methanesulfonamide respectively N,N′-(1S,2S)-1,2-cyclohexanediylbis[1,1,1-trifluoro]-methanesulfonamide, Ti(iPrO)₄ and a suitable solvent, such as for example toluene.

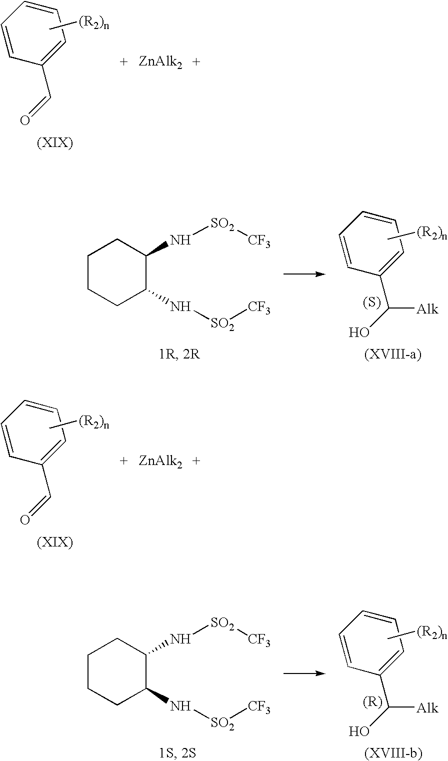

Intermediates of formula (V) can be prepared by reacting an intermediate of formula (XX) wherein W₅ represents a suitable leaving group, such as for example halo, e.g. chloro and the like, with an intermediate of formula (IX) and an intermediate of formula (XXI), in the presence of a suitable base, such as for example N,N-diisopropylethanamine, and a suitable solvent, such as for example tetrahydrofuran.

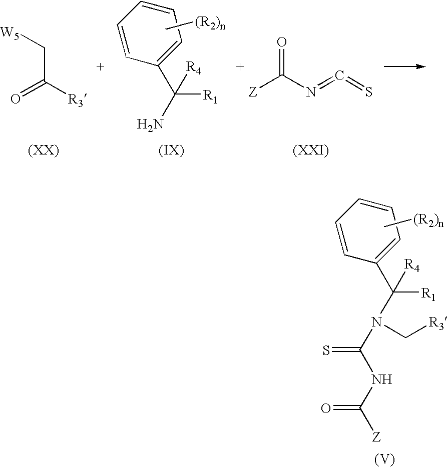

Intermediates of formula (VI) can be prepared by reacting an intermediate of formula (XXII) wherein W₆ represents a suitable leaving group, such as for example halo, e.g. chloro, with an intermediate of formula (XXIII) in the presence of a suitable solvent, such as for example tetrahydrofuran, optionally in the presence of a suitable base, such as for example N,N-diethylethanamine.

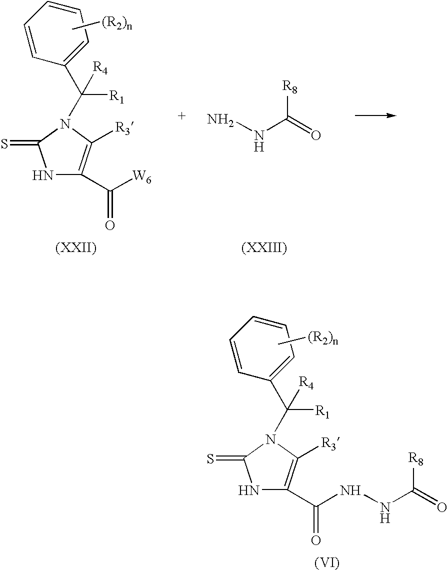

Intermediates of formula (XXII) wherein W₆ represents chloro, said intermediates being represented by formula (XXII-a), can be prepared by reacting an intermediate of formula (XXIV) with SOCl₂ optionally in the presence of a suitable solvent, such as for example methylene chloride.

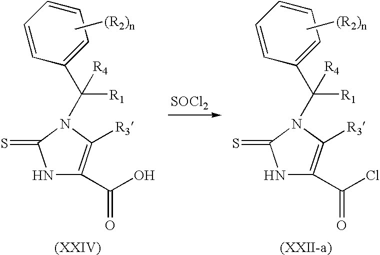

Intermediates of formula (XXIV) can be prepared by reacting an intermediate of formula (III) with an intermediate of formula (XXVI) in the presence of KSCN, NaOC(CH₃)₃, a suitable acid, such as for example hydrochloric acid, and a suitable solvent, such as for example tetrahydrofuran. This reaction also leads to the preparation of intermediates of formula (XXV).

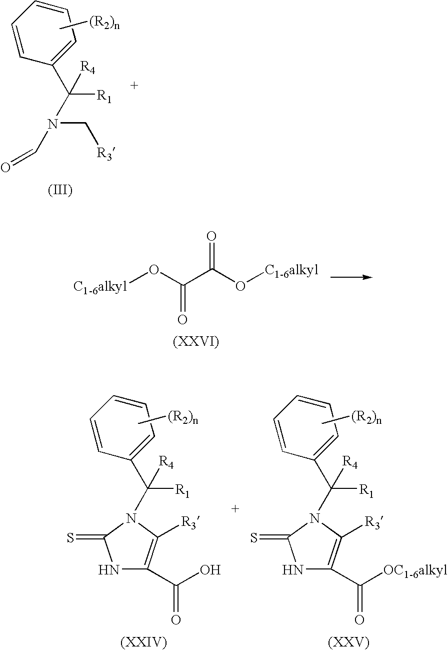

Intermediates of formula (XXIV) can also be prepared by hydrolyzing an intermediate of formula (XXV) in the presence of a suitable base, such as for example sodium hydroxide, in the presence of a suitable solvent, such as an alcohol, e.g. methanol and water. Intermediates of formula (XXIV) may also be prepared by hydrolysis of an intermediate of formula (XXV) in the presence of a suitable acid, such as for example trifluoroacetic acid, in the presence of a suitable solvent, such as for example methylene chloride.

In the preparation of the compounds of the present invention, interesting intermediates are intermediates of formula (IX) 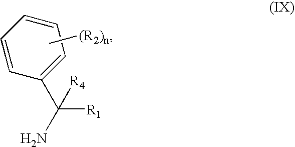
a N-oxide, a pharmaceutically acceptable addition salt, a quaternary amine or stereochemically isomeric form thereof.

As already indicated hereinabove, the intermediates of formula (IX) may contain a chiral center at the carbon atom carrying the R₁ and R₄ substituent depending on the substituents representing R₁ and R₄. In case said carbon atom represents a chiral center, a preferred embodiment of the intermediates of formula (IX) are those intermediates wherein the intermediate is stereospecific, i.e. wherein the intermediate has the (R) or (S) configuration at the carbon atom carrying the R₁ and R₄ substituent (intermediates of formula (IX-b). Particularly preferred are those intermediates of formula (IX-b) which have the (S) configuration (intermediates of formula (IX-b-1).

Thus the present invention also relates to intermediates of formula (IX-b-1) 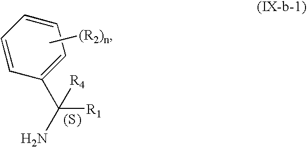
a N-oxide, a pharmaceutically acceptable addition salt or a quaternary amine thereof.

The present invention also relates to intermediates of formula (IX-b-1) provided that when n—2 and each R₂ is chloro and said two chloro substituents are placed in meta and para position, then R₁ is other than ethyl.

Another preferred embodiment are those intermediates of formula (IX-b-1) wherein each R₂ is independently selected from halo, C₁₋₆alkyl, C₁₋₆alkyloxy or polyhaloC₁₋₆alkyl. provided that when n=2 and each R₂ is chloro and said two chloro substituents are placed in meta and para position, then R₁ is other than ethyl.

A further embodiment are those intermediates of formula (IX-b-1) wherein n is 1, 2, or 3, in particular 2, provided that when n is 2 and each R₂ is chloro and said two chloro substituents are placed in meta and para position, then R₁ is other than ethyl.

Again another embodiment are those intermediates of formula (IX-b-1) wherein n is 2 and the two R₂ substituents are placed in meta and para position provided that when each R₂ is chloro, then R₁ is other than ethyl.

Another embodiment are those intermediates of formula (IX-b-1) as described hereinabove wherein R₄ is hydrogen.

Also interesting are those intermediates of formula (IX) and (IX-b-1) wherein R₁ is hydrogen, methyl, ethyl, n-propyl, methoxymethyl, cyclohexyl, cyclopropyl, dimethylaminomethyl, 2-thienyl, 3,4-dichlorophenyl, in particular methyl, ethyl, n-propyl, methoxymethyl, more in particular methyl, ethyl and n-propyl provided that when n is 2 and R₂ represents chloro, and said two chloro substituents are placed in meta and para position, and R₄ is hydrogen, then R₁ is other than ethyl, cyclopropyl phenyl, and provided that when n is 2 and R₂ represents chloro, and said two chloro substituents are placed in meta and para position, and R₄ is methyl, then R₁ is other than methyl and provided that when n is 2 and said two R₂ substituents are placed in meta and para position and R₂ in meta position is trifluoromethyl and R₂ in para position is fluoro, and R₄ is hydrogen, then R₁ is other than ethyl.

Also interesting are those intermediates of formula (IX) or (IX-b-1) as described hereinabove wherein R₂ is chloro, fluoro, or trifluoromethyl, in particular chloro or fluoro, more in particular fluoro.

Particularly interesting intermediates are those intermediates of formula (IX-a) or (IX-b-1) having the following formula 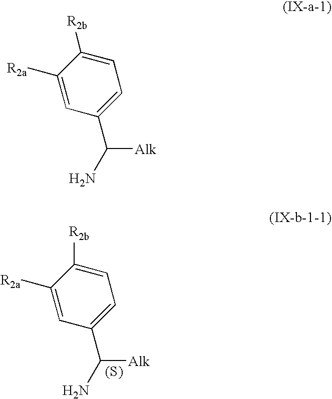
a N-oxide, a pharmaceutically acceptable addition salt or a quaternary amine thereof, wherein Alk is defined as hereinabove, i.e. Alk represents methyl, ethyl and n-propyl, and each R₂ₐ and R_{2b} independently represents chloro, fluoro or trifluoromethyl.

An interesting embodiment are those intermediates of formula (IX-a-1) provided that when R₂ₐ and R_{2b} are both chloro or when R₂ₐ is trifluoromethyl and R_{2b} is fluoro, then Alk is other than ethyl.

Further interesting intermediates of formula (IX-a-1) are those intermediates of formula (IX-a-1) provided that when R₂ₐ and R_{2b} are both chloro, then Alk is other than methyl, ethyl, n-propyl and provided that when R₂ₐ and R_{2b} are both fluoro or R₂ₐ is trifluoromethyl and R_{2b} is fluoro or R₂ₐ is fluoro and R_{2b} is trifluoromethyl then Alk is other than ethyl.

Also interesting are those intermediates of formula (IX-b-1-1) provided that when R₂ₐ and R_{2b} are both chloro, then Alk is other than ethyl.

A particular interesting intermediate of formula (IX-b-1-1) is that intermediate wherein R₂ₐ and R_{2b} are both fluoro and Alk represents ethyl. i.e. a compound of formula (IX-b-1-1-a).

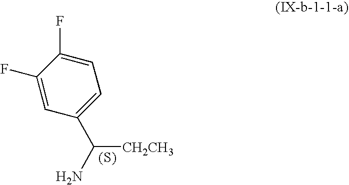

The compounds of formula (I) and (I′) show CCR2 receptor antagonistic properties.

The C—C chemokine receptor 2 (CCR2) and its ligand monocyte chemoattractant (chemotactic) protein (MCP-1; in new chemokine nomenclature also called CCL2) are recognized to be implicated in both acute and chronic inflammatory processes.

Chemokines (contraction of “chemotactic cytokines”) are most important regulators of leukocyte trafficking. This biological role is exerted by interacting—on target cells—with seven-transmembrane-domain receptors that are coupled to heterodimeric G proteins. Chemokines are mainly grouped into 2 major families (C—C or C—X—C family) dependent on the presence of an amino acid (represented by X) between the two conserved cysteine residues (represented by C) near the amino terminus. In general, chemokines from the C—C family attract monocytes, macrophages, T cells and NK cells.

A chemokine, which acts through the CCR2 receptor, is MCP-1 as indicated above. Therefore, the CCR2 receptor is also known as the MCP-1 receptor. MCP-2, MCP-3 and MCP-4 may also act, at least in part, through this receptor.

It is recognized that the CCR2 receptor and MCP-1 play a role in the pathophysiology of various inflammatory diseases. Therefore, CCR2 receptor antagonists, which block the CCR2 receptor, have potential as pharmaceutical agents to combat inflammatory conditions such as arthritis, osteoarthritis, rheumatoid arthritis, glomerulonephritis, diabetic nephropathy, lung fibrosis, idiopathic pulmonary fibrosis, sarcoidosis, vasculitis, hepatitis, nonalcoholic steatohepatitis, inflammatory conditions of the brain such as Alzheimer's disease, restenosis, alveolitis, asthma, allergic rhinitis, allergic conjunctivitis, atherosclerosis, psoriasis, delayed-type hypersensitivity reactions of the skin, inflammatory bowel disease, acute or chronic brain inflammation, e.g. multiple sclerosis, autoimmune encephalomyelitis, chronic obstructive pulmonary disease (COPD), uveitis, dermatitis, atopic dermatitis. CCR2 receptor antagonists may also be useful to treat autoimmune diseases such as diabetes or transplant rejection, stroke, reperfusion injury, ischemia, cancer, myocardial infraction, pain, in particular neuropathic pain.

The compounds of the present invention may also be used to inhibit the entry of Human Immunodeficiency Virus (HIV) into monocytes and lymphocytes, thereby having a therapeutic role in the treatment of AIDS (Acquired Immunodeficiency Syndrome).

The CCR2 receptor exists in two isoforms, namely the CCR2A and the CCR2B receptor.

Due to their CCR2 receptor antagonistic activity, in particular their CCR2B receptor antagonistic activity, the compounds of formula (I), their N-oxides, pharmaceutically acceptable addition salts, quaternary amines, polymorphic forms or stereochemically isomeric forms are useful in the treatment or prevention, in particular for the treatment, of diseases or conditions mediated through the activation of the CCR2 receptor, in particular the CCR2B receptor. Diseases or conditions related to an activation of the CCR2 receptor comprise inflammatory conditions such as arthritis, osteoarthritis, rheumatoid arthritis, glomerulonephritis, diabetic nephropathy, lung fibrosis, idiopathic pulmonary fibrosis, sarcoidosis, vasculitis, hepatitis, nonalcoholic steatohepatitis, inflammatory conditions of the brain such as Alzheimer's disease, restenosis, alveolitis, asthma, allergic rhinitis, allergic conjunctivitis, atherosclerosis, psoriasis, delayed-type hypersensitivity reactions of the skin, inflammatory bowel disease, acute or chronic brain inflammation, e.g. multiple sclerosis, autoimmune encephalomyelitis, chronic obstructive pulmonary disease (COPD), uveitis, dermatitis, atopic dermatitis, autoimmune diseases such as diabetes or transplant rejection, stroke, reperfusion injury, ischemia, cancer, myocardial infraction, pain (neuropathic pain). In particular, the compounds of formula (I) are useful in the treatment or prevention of inflammatory diseases and autoimmune diseases, especially rheumatoid arthritis, atherosclerosis, multiple sclerosis, inflammatory bowel disease and chronic obstructive pulmonary disease (COPD). The compounds of formula (I) are also of particular interest in the treatment or prevention of psoriasis, asthma, rheumatoid arthritis or pain (neuropathic pain), more in particular psoriasis, asthma or rheumatoid arthritis.

In view of the above-described pharmacological properties, the compounds of formula (I), their N-oxides, pharmaceutically acceptable addition salts, quaternary amines and stereochemically isomeric forms, may be used as a medicine. In particular, the present compounds can be used for the manufacture of a medicament for treating or preventing diseases mediated through activation of the CCR2 receptor, in particular the CCR2B receptor. More in particular, the compounds of the invention can be used for the manufacture of a medicament for treating or preventing inflammatory diseases, especially rheumatoid arthritis, atherosclerosis, multiple sclerosis, inflammatory bowel disease and chronic obstructive pulmonary disease (COPD). The compounds of the invention can also in particular be used for the manufacture of a medicament for treating or preventing psoriasis, asthma, rheumatoid arthritis or pain (neuropathic pain), more in particular psoriasis, asthma or rheumatoid arthritis.

In view of the utility of the compounds of formula (I), there is provided a method of treating warm-blooded animals, including humans, suffering from or a method of preventing warm-blooded animals, including humans, to suffer from diseases mediated through activation of the CCR2 receptor, in particular mediated through the CCR2B receptor. Said methods comprise the administration of an effective amount of a compound of formula (I), a N-oxide form, a pharmaceutically acceptable addition salt, a quaternary amine, a polymorphic form or a possible stereoisomeric form thereof, to warm-blooded animals, including humans.

The blockade of the CCR2 receptor by the present compounds of formula (I) inhibits the normal function of MCP-1. Therefore, the present compounds can also be described as MCP-1 inhibitors and hence can be used to prevent or treat diseases mediated through MCP-1.

The present invention also provides compositions for preventing or treating diseases mediated through activation of the CCR2 receptor, in particular the CCR2B receptor. Said compositions comprise a therapeutically effective amount of a compound of formula (I) and a pharmaceutically acceptable carrier or diluent.

The compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations, which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

The compounds of the present invention may also be administered via inhalation or insufflation by means of methods and formulations employed in the art for administration via this way. Thus, in general the compounds of the present invention may be administered to the lungs in the form of a solution, a suspension or a dry powder. Any system developed for the delivery of solutions, suspensions or dry powders via oral or nasal inhalation or insufflation are suitable for the administration of the present compounds.

The compounds of the present invention may also be topically administered in the form of drops, in particular eye drops. Said eye drops may be in the form of a solution or a suspension. Any system developed for the delivery of solutions or suspensions as eye drops are suitable for the administration of the present compounds.

It is especially advantageous to formulate the aforementioned pharmaceutical compositions in unit dosage form for ease of administration and uniformity of dosage.

Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The compounds of formula (I) may also be used in combination with other conventional anti-inflammatory or immunosuppressive agents, such as steroids, cyclooxygenase-2 inhibitors, non-steroidal-anti-inflammatory drugs, TNF-α antibodies, such as for example acetyl salicylic acid, bufexamac, diclofenac potassium, sulindac, diclofenac sodium, ketorolac trometamol, tolmetine, ibuprofen, naproxen, naproxen sodium, tiaprofen acid, flurbiprofen, mefenamic acid, nifluminic acid, meclofenamate, indomethacin, proglumetacine, ketoprofen, nabumetone, paracetamol, piroxicam, tenoxicam, nimesulide, fenylbutazon, tramadol, beclomethasone dipropionate, betamethasone, beclamethasone, budesonide, fluticasone, mometasone, dexamethasone, hydrocortisone, methylprednisolone, prednisolone, prednisone, triamcinolone, celecoxib, rofecoxib, valdecoxib, infliximab, leflunomide, etanercept, CPH 82, methotrexate, sulfasalazine, antilymphocytory immunoglobulines, antithymocytory immunoglobulines, azathioprine, cyclosporine, tacrolimus substances, ascomycin, rapamycin, muromonab-CD3.

Thus, the present invention also relates to the combination of a compound of formula (I) and another anti-inflammatory or immunosuppressive agent. Said combination may be used as a medicine. The present invention also relates to a product containing (a) a compound of formula (I), and (b) another anti-inflammatory or immunosuppressive compound, as a combined preparation for simultaneous, separate or sequential use in the treatment of diseases mediated through activation of the CCR2 receptor, in particular mediated through the CCR2B receptor. The different drugs in such products may be combined in a single preparation together with pharmaceutically acceptable carriers. Alternatively, such products may comprise, for example, a kit comprising a container with a suitable composition containing a compound of formula (I) and another container with a composition containing another anti-inflammatory or immunosuppressive compound. Such a product may have the advantage that a physician can select on the basis of the diagnosis of the patient to be treated the appropriate amounts of each component and the sequence and timing of the administration thereof.

The following examples are intended to illustrate the present invention.

### EXPERIMENTAL PART

Hereinafter “RT” means room temperature, “THF” means tetrahydrofuran, “DIPE” means diisopropylether, “TFA” means trifluoroacetic acid, “DBU” means 2,3,4,6,7,8,9,10-octahydropyrimido[1,2-a]azepine and “DMF” means N,N-dimethylformamide.

### A. Preparation of the Intermediate Compounds

### Example A1

a. Preparation of Intermediate 1

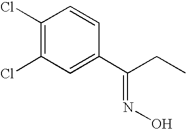

A solution of Na₂CO₃ (part of 0.52 mol) in H₂O (150 ml) was added to a stirring mixture of 1-(3,4-dichlorophenyl)-1-propanone (0.345 mol) in ethanol, p.a. (150 ml), then the remainder of Na₂CO₃ was added and hydroxylamine monohydrochloride (0.345 mol) was added portionwise while stirring vigorously. The reaction mixture was heated to reflux temperature and extra H₂O (75 ml) was added, then the resulting mixture was stirred and refluxed for 6 hours. Extra hydroxylamine monohydrochloride (2.4 g) was added and the mixture was refluxed further for 18 hours. Again extra hydroxylamine monohydrochloride (3 g) was added; the reaction mixture was refluxed for 24 hours and stirred for 2 days at room temperature. The solids were filtered off, washed with EtOH/H₂O (1/1) and dried (vacuum, stream of air) at 56° C. Yield: 71.8 g of intermediate 1 (95.4%).
b. Preparation of Intermediate 2 and 3

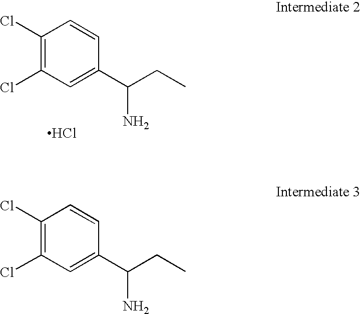

A mixture of intermediate 1 (0.3 mol) in CH₃OH/NH₃ (7 N) (500 ml) was hydrogenated at 14° C. with Raney Nickel (cat. quant.) as a catalyst in the presence of thiophene solution (6 ml). After uptake of H₂ (2 equiv.), the catalyst was filtered off and the filtrate was evaporated, then co-evaporated 2 times with toluene. The residue was stirred in boiling 2-propanol (250 ml) and the mixture was filtered off hot. The filtrate was allowed to reach room temperature and HC1/2-propanol (6N, 150 ml) was added slowly while stirring vigorously. The solvent was evaporated and the residue was stirred in DIPE, then filtered off, washed and dried (vacuum) at 60° C. Yield: 53 g of intermediate 2 (73.4%). A part of this fraction was converted into its free base: Intermediate 2 (18.0 g) was stirred in CH₂Cl₂ (200 ml) and a 15% aqueous K₂CO₃ solution was added, then the resulting mixture was stirred for 1 hour and a 50% NaOH solution was added to increase the pH. The organic layer was separated, washed with H₂O, dried (MgSO₄), filtered off and the solvent was evaporated. Yield: 51 g of intermediate 3.

### Example A2

a. Preparation of Intermediate 4

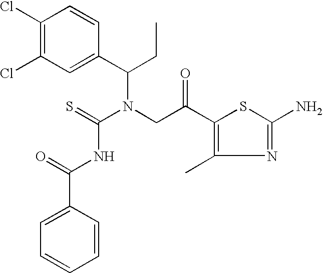

Intermediate 3 (prepared according to A1.b) (0.00208 mol) was dissolved in THF (15 ml) and N-ethyl-N-(1-methylethyl)-2-propanamine (0.0052 mol) was added, followed by 1-(2-amino-4-methyl-5-thiazolyl)-2-bromo-ethanone monohydrobromide (0.00104 mol). The reaction mixture was stirred at room temperature for 18 hours and benzoyl isothiocyanate (0.003 mol) was added, then the mixture was stirred for 2 hours at room temperature and poured out into ice-water (60 ml). The product was extracted with CH₂Cl₂ and then the organic layer was separated, dried (MgSO₄) and filtered off. Finally, the solvent was evaporated. Yield: 1.38 g of intermediate 4.
b. Preparation of Intermediate 5

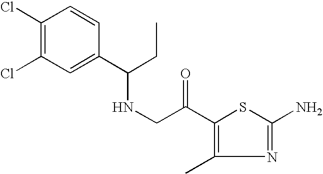

Intermediate 3 (prepared according to A1.b) (208 mg) was dissolved in THF, p.a., dried on molecular sieves, (15 ml) and then N-ethyl-N-(1-methylethyl)-2-propanamine (0.0016 mol) was added followed by 1-(2-amino-4-methyl-5-thiazolyl)-2-bromo-ethanone monohydrobromide (0.00095 mol). The reaction mixture was stirred for 18 hours at room temperature and the resulting precipitate was filtered off, washed with THF and dried in vacuum. Yield: 330 mg of intermediate 5.

### Example A3

a. Preparation of Intermediate 6

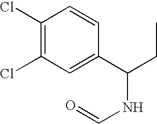

A solution of 1-(3,4-dichlorophenyl)-1-propanone (0.0748 mol) in formamide (45 ml) and formic acid (30 ml) was stirred and refluxed for 22 hours and then the reaction mixture was allowed to reach room temperature. The mixture was warmed to 50° C., poured out into ice water (400 ml) and extracted with EtOAc. The organic layer was separated, washed with H₂O, with a saturated aqueous NaHCO₃ solution and again with H₂O and with brine, then dried (MgSO₄) and filtered off. The solvent was evaporated and the residual oil was left to stand overnight. The resulting solids were filtered off, stirred in DIPE, filtered off again, washed and dried (vacuum) at 50° C. Yield: 14.0 g of intermediate 6 (80.6%).
b. Preparation of Intermediate 7

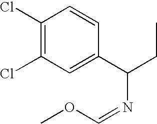

A solution of intermediate 6 (0.0032 mol) in CH₂Cl₂ p.a. (5 ml) was stirred at room temperature under N₂ and then a solution of trimethyloxonium tetrafluoroborate (0.00331 mol) in CH₂Cl₂ p.a. (5 ml) was added. The reaction mixture was stirred at room temperature for 24 hours and was slowly poured out into an aqueous NaOH solution with ice, then CH₂Cl₂ was added. The separated organic layer was dried over NaOH-pellets, filtered off and the solvent was evaporated, yielding intermediate 7 which was used in the next step.
c. Preparation of Intermediate 8

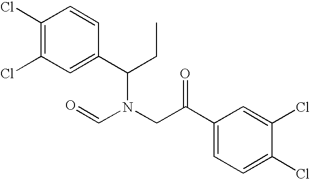

2-Bromo-1-(3,4-dichlorophenyl)ethanone (0.00284 mol) was added to a stirring solution of intermediate 7 (0.00569 mol) in dry CH₃CN, p.a. (35 ml) under N₂ and then the solution was stirred and refluxed for 24 hours. The reaction mixture was left to stand over the weekend and the solvent was evaporated. The residue was purified by column chromatography over silica gel (eluent gradient: CH₂Cl₂/CH₃OH 100/0->99.5/0.5->99/1). The product fractions were collected and the solvent was evaporated. Yield: 1.1 g of fraction 1. A part (0.55 g) of this fraction was purified by reversed phase high-performance liquid chromatography (CH₃CN). The product fractions were collected and the organic solvent was evaporated. The aqueous concentrate was extracted with CH₂Cl₂ and the separated organic layer was evaporated. Yield: 0.12 g of intermediate 8.

### Example A4

a. Preparation of intermediate 9

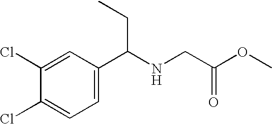

A solution of intermediate 2 (prepared according to A1.b) (0.0748 mol) and chloro acetic acid methyl ester (0.08 mol) in DMF, p.a., dried on molecular sieves, (150 ml) was stirred at room temperature under N₂ and Et₃N (0.224 mol) was slowly added, then the reaction mixture was stirred for 20 hours at room temperature and extra chloro acetic acid methyl ester (3.3 ml) was added. The mixture was stirred for another 20 hours at room temperature and again extra chloro acetic acid methyl ester (2 ml) was added. The resulting mixture was stirred for 24 hours and then the solids were filtered off and washed with DMF. Et₂O (800 ml) was added and the mixture washed 3 times with H₂O (500 ml). The organic layer was separated, dried (MgSO₄), filtered off and the solvent was evaporated, then co-evaporated with toluene. The residual oil (23.4 g) was filtered over silica gel (eluent: CH₂Cl₂/CH₃OH 99/1). The product fractions were collected and the solvent was evaporated, finally co-evaporated with toluene. Yield: 20.6 g of intermediate 9 (99.7%).
b. Preparation of Intermediate 10

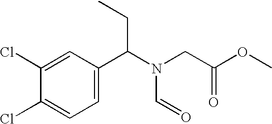

A solution of formic acid (7.5 ml) and intermediate 9 (0.0746 mol) in xylene, p.a. (225 ml) was stirred and refluxed for 4 hours and then the reaction mixture was allowed to reach room temperature. The mixture washed 2 times with H₂O (2×200 ml), with a saturated aqueous NaHCO₃ solution (200 ml) and with brine (200 ml), then the separated organic layer was dried (MgSO₄) and filtered off. Finally, the solvent was evaporated. Yield: 21.3 g of intermediate 10 (93.9%)

### Example A5

a. Preparation of Intermediate 11 and 20

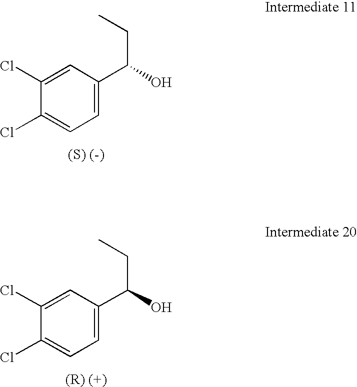

A mixture of N,N′-(1R,2R)-1,2-cyclohexanediylbis[1,1,1-trifluoromethanesulfonamide] (0.005 mol) and Ti(i-PrO)₄ (0.030 mol) in toluene (q.s.) was degassed and placed under Ar-flow, then the reaction mixture was stirred for 20 minutes at 40° C. and cooled to −78° C. Et₂Zn (0.030 mol) was added dropwise and after 20 minutes, a mixture of 3,4-dichlorobenzaldehyde (0.0250 mol) in toluene (q.s.) was added dropwise. The reaction mixture was allowed to reach 0° C. The mixture was stirred overnight at room temperature, then quenched with HCl (2N). This mixture was extracted with CH₂Cl₂. The separated organic layer washed, dried, filtered and the solvent evaporated. The residue was purified by column chromatography over silica gel (eluent CH₂Cl₂/CH₃OH 98/2). The product fractions were collected and the solvent was evaporated. Yield: 5.1 g of intermediate 11. Intermediate 20 can be prepared by the above reaction by using N,N′-(1S,2S)-1,2-cyclohexanediylbis[1,1,1-trifluoromethanesulfonamide] as catalyst.
b-1). Preparation of Intermediate 12

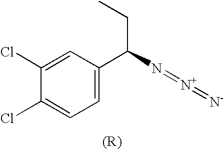

A mixture of intermediate 11 (0.025 mol; prepared according to A5.a) and diphenylphosphoryl azide (0.030 mol) in toluene (50 ml) was stirred at 0° C. and 2,3,4,6,7,8,9,10-octahydropyrimido[1,2-a]azepine (0.030 mol) was added. The reaction mixture was stirred for 2 hours at 0° C., then stirred overnight at room temperature. The mixture was diluted with water and toluene. The organic layer was separated, washed once with water, once with 5% HCl, and the solvent was evaporated, yielding intermediate 12 (quantitative yield; used in next reaction step).
b-2). Preparation of Intermediate 21

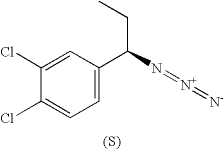

A mixture of intermediate 20 (prepared according to A5.a) (0.127 mol) and diphenylphosphoryl azide (0.153 mol) in toluene (q.s.) was stirred at 0° C. 2,3,4,6,7,8,9,10-octahydropyrimido[1,2-a]azepine (0.153 mol) was added dropwise and the reaction mixture was stirred for 1 hour at 0° C., then for 2 hours at room temperature, then for 3 hours at 50° C. The reaction mixture was cooled, washed with water, with 0.5 M HCl, with water, dried, filtered and the solvent was evaporated. The residue was purified by flash column chromatography over silica gel (eluent: CH₂Cl₂/CH₃OH 99.5/0.5). The product fractions were collected and the solvent was evaporated. Yield: 23.5 g of intermediate 21.
c-1. Preparation of Intermediate 13

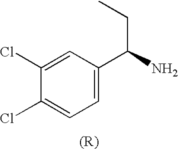

A mixture of intermediate 12 (0.025 mol; prepared according to A5.b-1), triphenylphosphine (0.027 mol) in THF (70 ml) and H₂O (20 ml) was stirred overnight at room temperature. The solvent was evaporated The residue was treated with 10% HCl. The acidic layer washed with DIPE, then alkalized, followed by an extraction with CH₂Cl₂. The separated organic layer was dried, filtered and the solvent evaporated. The residue was purified by column chromatography over silica gel. The product fractions were collected and the solvent was evaporated. Yield: 1.1 g of intermediate 13.
c-2. Preparation of Intermediate 22

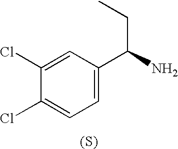

A mixture of intermediate 21 (prepared according to A5.b-2) (0.122 mol; 23.5 g) in methanol (q.s.) was hydrogenated at 50° C. with Pt/C₅% (5 g) as a catalyst. After uptake of H₂, the catalyst was filtered off and the filtrate was evaporated. Yield: 21 g of intermediate 22.
d. Preparation of Intermediate 14

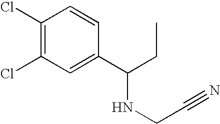

A solution of intermediate 3 (prepared according to A1.b) (0.0116 mol) in Et₃N (0.013 mol) and DMF, p.a., dried on molecular sieves, (20 ml) was stirred on an ice bath. A solution of chloro acetonitrile (0.0128 mol) in DMF, p.a., dried on molecular sieves, (2.5 ml) was added dropwise. The reaction mixture was stirred at room temperature for 6 hours. More chloro acetonitrile (0.0063 mol) in DMF, p.a., dried on molecular sieves, (1 ml) was added dropwise. The reaction mixture was stirred for another 24 hours. More chloro acetonitrile (0.0063 mol) in DMF, p.a., dried on molecular sieves, (1 ml) was added dropwise and the reaction mixture was stirred for another 24 hours. More Et₃N (1 ml) was added, then more chloro acetonitrile (0.0079 mol) in DMF, p.a., dried on molecular sieves, (1 ml) was added dropwise. The reaction mixture was stirred for 20 hours. The precipitate was filtered off. The filtrate was poured out into Et₂O (200 ml) and washed with H₂O/NaHCO₃ (10%;100 ml) and H₂O (2×). The separated organic layer was dried (MgSO₄), filtered and the solvent was evaporated and co-evaporated with toluene. The residue was purified over silica gel (eluent: CH₂Cl₂/MeOH 99:1). The desired fractions were collected and the solvent was evaporated and coevaporated with toluene. Yield: 2.3 g of intermediate 14 (81.6%).
e. Preparation of Intermediate 15

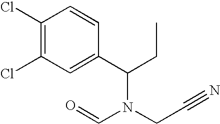

A mixture of intermediate 14 (0.00946 mol) and n-butyl formate (15 ml) was stirred and refluxed for 4 days. The solvent was evaporated, then co-evaporated with toluene. Yield: 2.68 g of intermediate 15.

### Example A6

a. Preparation of Intermediate 16 and 17

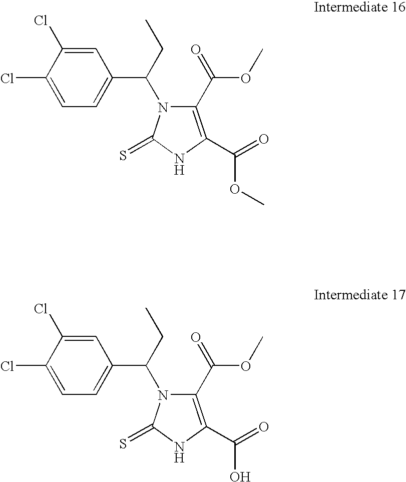

A solution of intermediate 10 (prepared according to A4.b) (0.0618 mol) and dimethyl ethanedioic acid ester (0.11 mol) in THF, p.a., dried on molecular sieves, (100 ml) was stirred under N₂-atmosphere, then 2-methyl-2-propanol sodium salt (0.066 mol) was added and the reaction mixture was stirred at room temperature for 18 hours and another 24 hours. Finally the mixture was stirred at 60° C. for 4 hours. Extra 2-methyl-2-propanol sodium salt (4 g) and extra dimethyl ethanedioic acid ester (6 g) were added and the reaction mixture was stirred over the weekend at room temperature. The solvent was evaporated, the residue was dissolved in H₂O (250 ml) and washed 2 times with Et₂O. The aqueous layer was separated and CH₃OH (200 ml), potassium thiocyanic acid salt (10 g) and HCl (36%, p.a.) (q.s.) were added, then the mixture was stirred for 18 hours at 60° C. The solvent was partly evaporated and the concentrate was extracted with CH₂Cl₂. The organic layer was separated, dried (MgSO₄), filtered and the solvent was evaporated. The residue (5 g) was purified by filtration over silica gel (eluent: CH₂Cl₂/CH₃OH 99/1). The product fractions were collected and the solvent was evaporated. The residue was triturated under Hexane, filtered off, washed, then dried (vacuum, 50° C.). Yield: 5.2 g of intermediate 16. The fractions containing a side-product were combined and the solvent was evaporated, then co-evaporated with hexane/DIPE. The residue was stirred in Et₂O/Hexane and the resulting precipitate was filtered off, washed with hexane, then dried (vacuum, 50° C.). Yield: 0.28 g of intermediate 17.
b. Preparation of Intermediate 18

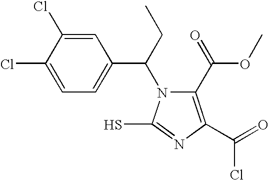

A mixture of intermediate 17 (0.000257 mol) in thionyl chloride (5 ml) was stirred overnight at room temperature, then the solvent was evaporated and co-evaporated with toluene (p.a.), yielding intermediate 18 used in the next step.
c. Preparation of Intermediate 19

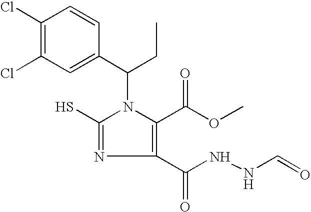

A solution of intermediate 18 (0.000257 mol) in THF, p.a., dried on molecular sieves, (10 ml) was stirred under N₂ and then hydrazinecarboxaldehyde (0.0125 mol) was added. The reaction mixture was stirred at room temperature for 1 hour and the solvent was evaporated. The residue was stirred in H₂O (10 ml) and CH₂Cl₂/CH₃OH (15 ml, 95/5). The organic layer was separated, washed with HCl (1N), dried (MgSO₄), filtered off and the solvent was evaporated, then co-evaporated with toluene. Yield: 800 mg of intermediate 19.

### Example A7

a-1. Preparation of Intermediate 24

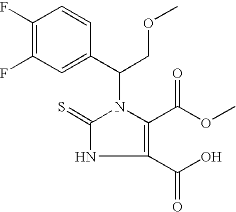

A mixture of 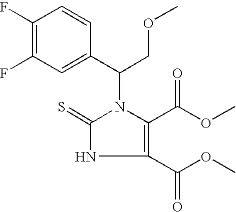
(intermediate 23 prepared according to A6.a) (0.0015 mol) and 1N NaOH (0.030 mol) in H₂O (20 ml) and methanol (50 ml) was stirred for 20 hours at room temperature, then extra 1N NaOH (10 ml) was added and the reaction mixture was stirred for 5 hours at room temperature. Half of the solvent was evaporated and 1N HCl (40 ml) was added and the precipitated product was extracted with CH₂Cl₂. The organic layer was separated, dried, filtered off and the solvent was evaporated. Yield: 5.6 g of intermediate 24.
a-2. Preparation of Intermediate 28

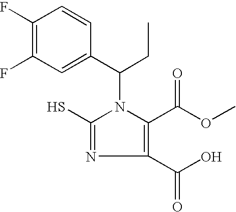

A mixture of 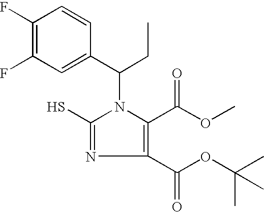
(intermediate 27 prepared according to A6.a) (0.0012 mol) in TFA (2 ml) and CH₂Cl₂ (70 ml) was stirred for 1 day at room temperature and then the reaction mixture was concentrated, yielding intermediate 28.
b. Preparation of Intermediate 25

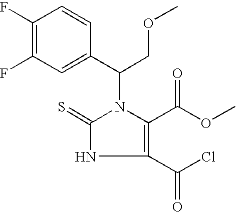

A mixture of intermediate 24 (prepared according to A7.a-1) (0.0075 mol) and SOCl₂ (0.015 mol) in CH₂Cl₂ (50 ml) was stirred and refluxed on an oil bath for 3 hours and then the solvent was evaporated. The residue was dissolved in toluene and the solvent was evaporated again. Yield: 2.9 g of intermediate 25.
c. Preparation of Intermediate 26

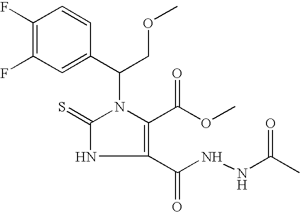

A mixture of hydrazide acetic acid (0.011 mol) and Et₃N (0.015 mol) in THF (30 ml) was stirred and cooled on an ice bath at 0-5° C. and then a mixture of intermediate 25 (prepared according to A7.b) (0.0075 mol) in THF (20 ml) was added dropwise over 30 minutes at 0-5° C. The reaction mixture was stirred for 1 hour at 0-5° C. and then H₂O was added and the mixture was extracted with CH₂Cl₂. The organic layer was separated, dried, filtered off and the solvent was evaporated. The obtained residue was filtered over silica gel (eluent: CH₂Cl₂/CH₃OH 97/3), then the product fractions were collected and the solvent was evaporated. Yield: 1.25 g of intermediate 26.

### Example A8

a. Preparation of Intermediate 30

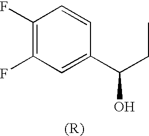

A solution of N,N′-(1S,2S)-1,2-cyclohexanediylbis[1,1,1-trifluoromethanesulfonamide] (catalytic quantity) and tetrakis(2-propanolato)titanium (0.415 mol) in toluene (p.a) (500 ml) was degassed under Argon and then stirred for 20 minutes at 1 40° C. This mixture was cooled on a 2-propanol/CO₂ bath to −78° C. and diethylzinc (0.415 mol) was added dropwise over 15 minutes. The resulting mixture was stirred for 15 minutes at −78° C. and then a solution of 3,4-difluorobenzaldehyde (0.345 mol) in toluene (p.a.) (q.s.) was added dropwise over 20 minutes. The reaction mixture was stirred further for 30 minutes at −78° C. and was then allowed to slowly reach 0° C. The mixture was quenched with 1N HCl and extracted with CH₂Cl₂. Both organic layer and aqueous layer were filtered over dicalite and the organic layer in the obtained filtrate was separated, then washed with H₂O, dried (MgSO₄) and filtered off. Finally, the solvent was evaporated. Yield: 57.7 g of intermediate 30.
b. Preparation of Intermediate 31

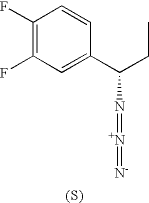

A solution of intermediate 30 (0.331 mol) and diphenylphosphoryl azide (0.4 mol) in toluene (p.a.) (500 ml) was stirred under N₂ on an ice bath at 0° C., then DBU (0.4 mol) was added dropwise and the reaction mixture was stirred for 1 hour at 0° C. The mixture was allowed to reach room temperature and was then stirred for 1 hour at 45° C. and overnight at room temperature. The resulting mixture was poured out into H₂O (500 ml) and extracted with Et₂O (500 ml). The organic layer was separated, dried (MgSO₄), filtered off and the solvent was evaporated. The residue was filtered over silica gel (eluent: CH₂Cl₂ 100%). The product fractions were collected and then the solvent was evaporated and co-evaporated with Toluene. Yield: 57.2 g of intermediate 31.
c. Preparation of Intermediate 32

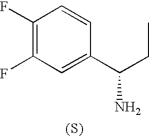

A mixture of intermediate 31 (0.29 mol) in CH₃OH (600 ml) was hydrogenated with Pd/C 10% (2 g) as a catalyst. After uptake of H₂ (1 equivalent), the catalyst was filtered off and the filtrate was evaporated. The residue was dissolved in CH₂Cl₂ (300 ml) and then 1N HCl was slowly added while the solution was stirred vigorously on an ice bath. After this extraction, the aqueous layer was alkalised with a 50% NaOH solution to pH>10 and the resulting mixture was extracted with CH₂Cl₂. The organic layer was separated, dried (MgSO₄), filtered off and the solvent was evaporated. Yield: 40.0 g of intermediate 32.
d. Preparation of Intermediate 33

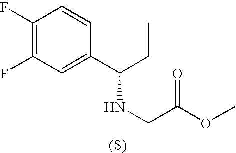

Brome acetic acid methyl ester (0.28 mol) was added to a stirring solution of intermediate 32 (0.233 mol) and N-ethyl-N-(1-methylethyl)-2-propanamine (0.466 mol) in THF (350 ml) and the reaction mixture was stirred for 5 days at room temperature. The resulting precipitate was filtered off, washed with THF and then the filtrate was evaporated. The obtained residue was stirred in CH₂Cl₂ and then washed with a half saturated aqueous NaHCO₃ solution and with water. The organic layer was separated, dried (MgSO₄), filtered off and then the solvent was evaporated and co-evaporated with toluene. Yield: intermediate 33.
e. Preparation of Intermediate 34

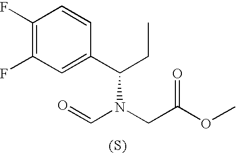

Formic acid (23 ml) was added to a stirring solution of intermediate 33 (0.23 mol) in xylene (p.a) (400 ml), then the reaction mixture was stirred and refluxed for 4 hours with a Dean-Stark apparatus. The mixture was allowed to reach room temperature and the solvent was evaporated. The residue was filtered over silica gel (eluent: CH₂Cl₂/CH₃OH 99/1). The product fractions with a purity >95% were collected and then the solvent was evaporated and co-evaporated with toluene. Yield: 15.2 g of intermediate 34.

### Example A9

a. Preparation of Intermediate 35

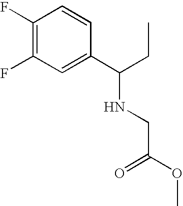

Following procedure was done 3×. [A mixture of 1-(3,4-difluorophenyl)-1-propanone (0.43 mol), glycine methyl ester, hydrochloride (0.5 mol) and KF (0.43 mol) in CH₃OH (700 ml) was hydrogenated at 50° C. (in Parr apparatus) with Pd/C 10% (5 g) as a catalyst in the presence of thiophene solution (2 ml). After uptake of H₂ (1 equivalent) was complete, the catalyst was filtered off and the filtrate was evaporated. The residue was stirred in water, then treated with NaHCO₃ (q.s.) and the product was extracted with CH₂Cl₂. The organic layer was separated, dried, filtered and the solvent evaporated. The residue was dissolved in 2-propanol (1000 ml) and converted into the hydrochloric acid salt with 6 N HCl/2-propanol. The precipitate was filtered off, washed with DIPE (to remove the 2-propanol), then stirred in water. CH₂Cl₂ was added and the mixture was treated with K₂CO₃ (q.s.). The layers were separated. The organic layer was dried, filtered and the solvent evaporated.] Yield: 221 g of intermediate 35.
b. Preparation of Intermediate 36

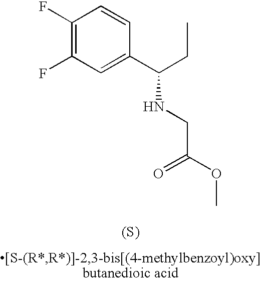

A mixture of intermediate 35 (0.9 mol) in 2-propanol (1500 ml) was stirred at room temperature. [S—(R*,R*)]-2,3-bis[(4-methylbenzoyl)oxy]-butanedioic acid (0.9 mol) was added in one portion. The reaction mixture was stirred for 2 hours at room temperature. The precipitate was filtered off and dissolved in refluxing 2-propanol (1500 ml). Then the mixture was stirred at room temperature, allowing the compound to precipitate again. The precipitate was filtered off, then re-dissolved in boiling 2-propanol. The mixture was allowed to cool to room temperature and the resulting precipitate was filtered off and dried. Yield: 231.4 g of intermediate 36 (96% (S)).
c. Preparation of Intermediate 37

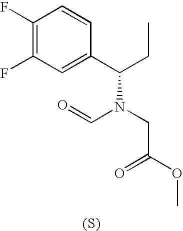

Intermediate 36 (370 g, 0.588 mol) was converted into the free base upon addition of NH₄OH/CH₂Cl₂. The product was extracted to give free base residue A*. A mixture of A* (132 g, 0.543 mol) in formic acid (150 ml) and xylene (750 ml) was stirred and refluxed for 5 hours at 120° C. The solvent was evaporated. The residue was stirred in water, treated with NaHCO₃ and then the product was extracted with CH₂Cl₂. The organic layer was separated, dried, filtered and the solvent evaporated. Yield: 146 g of intermediate 37 used in next reaction step, without further purification).

Table 1 lists intermediates of formula (IX) which can be prepared according to one of the above examples (Ex. No.).

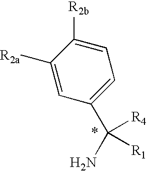

| | | | | | | Stereo- |
|---|---|---|---|---|---|---|
| | | | | | | chem |
| | | | | | | descrip- |
| Interm. | Ex.No. | R₂ₐ | R_{2b} | R₁ | R₄ | tor * |
| 1.1 | Alb | Cl | Cl | H | H | |
| 1.2 | Alb | Cl | Cl | CH₃ | H | (RS) |
| 1.3 | A5c-2) | Cl | Cl | CH₃ | H | (S) |
| 3 | Alb | Cl | Cl | CH₂CH₃ | H | (RS) |
| 22 | A5c-2) | Cl | Cl | CH₂CH₃ | H | (S) |
| 1.4 | Alb | Cl | Cl | CH₂CH₂CH₃ | H | (RS) |
| 1.5 | A5c-2) | Cl | Cl | CH₂CH₂CH₃ | H | (S) |
| 1.6 | Alb | Cl | Cl | phenyl | H | (RS) |
| 1.7 | Alb | Cl | Cl | 3,4-dichlorophenyl | H | (RS) |
| 1.8 | Alb | Cl | Cl | cyclohexyl | H | (RS) |
| 1.9 | Alb | Cl | Cl | cyclopropyl | H | (RS) |
| 1.10 | Alb | Cl | Cl | CH₂—N(CH₃)₂ | H | (RS) |
| 1.11 | Alb | Cl | Cl | CH₂—O—CH₃ | H | (RS) |
| 1.12 | Alb | Cl | Cl | 2-thienyl | H | (RS) |
| 2.1 | Alb | Cl | H | H | H | |
| 2.2 | Alb | Cl | H | CH₃ | H | (RS) |
| 2.3 | A5c-2) | Cl | H | CH₃ | H | (S) |
| 2.4 | Alb | Cl | H | CH₂CH₃ | H | (RS) |
| 2.5 | A5c-2) | Cl | H | CH₂CH₃ | H | (S) |
| 2.6 | Alb | Cl | H | CH₂CH₂CH₃ | H | (RS) |
| 2.7 | A5c-2) | Cl | H | CH₂CH₂CH₃ | H | (S) |
| 2.8 | Alb | Cl | H | phenyl | H | (RS) |
| 2.9 | Alb | Cl | H | 3,4-dichlorophenyl | H | (RS) |
| 2.10 | Alb | Cl | H | cyclohexyl | H | (RS) |
| 2.11 | Alb | Cl | H | cyclopropyl | H | (RS) |
| 2.12 | Alb | Cl | H | CH₂—N(CH₃)₂ | H | (RS) |
| 2.13 | Alb | Cl | H | CH₂—O—CH₃ | H | (RS) |
| 2.14 | Alb | Cl | H | 2-thienyl | H | (RS) |
| 3.1 | Alb | H | Cl | H | H | |
| 3.2 | Alb | H | Cl | CH₃ | H | (RS) |
| 3.3 | A5c-2) | H | Cl | CH₃ | H | (S) |
| 3.4 | Alb | H | Cl | CH₂CH₃ | H | (RS) |
| 3.5 | A5c-2) | H | Cl | CH₂CH₃ | H | (S) |
| 3.6 | Alb | H | Cl | CH₂CH₂CH₃ | H | (RS) |
| 3.7 | A5c-2) | H | Cl | CH₂CH₂CH₃ | H | (S) |
| 3.8 | Alb | H | Cl | phenyl | H | (RS) |
| 3.9 | Alb | H | Cl | 3,4-dichlorophenyl | H | (RS) |
| 3.10 | Alb | H | Cl | cyclohexyl | H | (RS) |
| 3.11 | Alb | H | Cl | cyclopropyl | H | (RS) |
| 3.12 | Alb | H | Cl | CH₂—N(CH₃)₂ | H | (RS) |
| 3.13 | Alb | H | Cl | CH₂—O—CH₃ | H | (RS) |
| 3.14 | Alb | H | Cl | 2-thienyl | H | (RS) |
| 4.1 | Alb | CH₃ | CH₃ | H | H | |
| 4.2 | Alb | CH₃ | CH₃ | CH₃ | H | (RS) |
| 4.3 | A5c-2) | CH₃ | CH₃ | CH₃ | H | (S) |
| 4.4 | Alb | CH₃ | CH₃ | CH₂CH₃ | H | (RS) |
| 4.5 | A5c-2) | CH₃ | CH₃ | CH₂CH₃ | H | (S) |
| 4.6 | Alb | CH₃ | CH₃ | CH₂CH₂CH₃ | H | (RS) |
| 4.7 | A5c-2) | CH₃ | CH₃ | CH₂CH₂CH₃ | H | (S) |
| 4.8 | Alb | CH₃ | CH₃ | phenyl | H | (RS) |
| 4.9 | Alb | CH₃ | CH₃ | 3,4-dichlorophenyl | H | (RS) |
| 4.10 | Alb | CH₃ | CH₃ | cyclohexyl | H | (RS) |
| 4.11 | Alb | CH₃ | CH₃ | cyclopropyl | H | (RS) |
| 4.12 | Alb | CH₃ | CH₃ | CH₂—N(CH₃)₂ | H | (RS) |
| 4.13 | Alb | CH₃ | CH₃ | CH₂—O—CH₃ | H | (RS) |
| 4.14 | Alb | CH₃ | CH₃ | 2-thienyl | H | (RS) |
| 5.1 | Alb | CH₃O | CH₃O | H | H | |
| 5.2 | Alb | CH₃O | CH₃O | CH₃ | H | (RS) |
| 5.3 | A5c-2) | CH₃O | CH₃O | CH₃ | H | (S) |
| 5.4 | Alb | CH₃O | CH₃O | CH₂CH₃ | H | (RS) |
| 5.5 | A5c-2) | CH₃O | CH₃O | CH₂CH₃ | H | (S) |
| 5.6 | Alb | CH₃O | CH₃O | CH₂CH₂CH₃ | H | (RS) |
| 5.7 | A5c-2) | CH₃O | CH₃O | CH₂CH₂CH₃ | H | (S) |
| 5.8 | Alb | CH₃O | CH₃O | phenyl | H | (RS) |
| 5.9 | Alb | CH₃O | CH₃O | 3,4-dichlorophenyl | H | (RS) |
| 5.10 | Alb | CH₃O | CH₃O | cyclohexyl | H | (RS) |
| 5.11 | Alb | CH₃O | CH₃O | cyclopropyl | H | (RS) |
| 5.12 | Alb | CH₃O | CH₃O | CH₂—N(CH₃)₂ | H | (RS) |
| 5.13 | Alb | CH₃O | CH₃O | CH₂—O—CH₃ | H | (RS) |
| 5.14 | Alb | CH₃O | CH₃O | 2-thienyl | H | (RS) |
| 6.1 | Alb | F | F | H | H | |
| 6.2 | Alb | F | F | CH₃ | H | (RS) |
| 6.3 | A5c-2) | F | F | CH₃ | H | (S) |
| 6.4 | Alb | F | F | CH₂CH₃ | H | (RS) |
| 6.5 | A5c- | F | F | CH₂CH₃ | H | (S) |
| | 2)/A8c | | | | | |
| 6.6 | Alb | F | F | CH₂CH₂CH₃ | H | (RS) |
| 6.7 | A5c-2) | F | F | CH₂CH₂CH₃ | H | (S) |
| 6.8 | Alb | F | F | phenyl | H | (RS) |
| 6.9 | Alb | F | F | 3,4-dichlorophenyl | H | (RS) |
| 6.10 | Alb | F | F | cyclohexyl | H | (RS) |
| 6.11 | Alb | F | F | cyclopropyl | H | (RS) |
| 6.12 | Alb | F | F | CH₂—N(CH₃)₂ | H | (RS) |
| 6.13 | Alb | F | F | CH₂—O—CH₃ | H | (RS) |
| 6.14 | Alb | F | F | 2-thienyl | H | (RS) |
| 7.1 | Alb | Br | Br | H | H | |
| 7.2 | Alb | Br | Br | CH₃ | H | (RS) |
| 7.3 | A5c-2) | Br | Br | CH₃ | H | (S) |
| 7.4 | Alb | Br | Br | CH₂CH₃ | H | (RS) |
| 7.5 | A5c-2) | Br | Br | CH₂CH₃ | H | (S) |
| 7.6 | Alb | Br | Br | CH₂CH₂CH₃ | H | (RS) |
| 7.7 | A5c-2) | Br | Br | CH₂CH₂CH₃ | H | (S) |
| 7.8 | Alb | Br | Br | phenyl | H | (RS) |
| 7.9 | Alb | Br | Br | 3,4-dichlorophenyl | H | (RS) |
| 7.10 | Alb | Br | Br | cyclohexyl | H | (RS) |
| 7.11 | Alb | Br | Br | cyclopropyl | H | (RS) |
| 7.12 | Alb | Br | Br | CH₂—N(CH₃)₂ | H | (RS) |
| 7.13 | Alb | Br | Br | CH₂—O—CH₃ | H | (RS) |
| 7.14 | Alb | Br | Br | 2-thienyl | H | (RS) |
| 8.1 | Alb | F | CF₃ | H | H | |
| 8.2 | Alb | F | CF₃ | CH₃ | H | (RS) |
| 8.3 | A5c-2) | F | CF₃ | CH₃ | H | (S) |
| 8.4 | Alb | F | CF₃ | CH₂CH₃ | H | (RS) |
| 8.5 | A5c-2) | F | CF₃ | CH₂CH₃ | H | (S) |
| 8.6 | Alb | F | CF₃ | CH₂CH₂CH₃ | H | (RS) |
| 8.7 | A5c-2) | F | CF₃ | CH₂CH₂CH₃ | H | (S) |
| 8.8 | Alb | F | CF₃ | phenyl | H | (RS) |
| 8.9 | Alb | F | CF₃ | 3,4-dichlorophenyl | H | (RS) |
| 8.10 | Alb | F | CF₃ | cyclohexyl | H | (RS) |
| 8.11 | Alb | F | CF₃ | cyclopropyl | H | (RS) |
| 8.12 | Alb | F | CF₃ | CH₂—N(CH₃)₂ | H | (RS) |
| 8.13 | Alb | F | CF₃ | CH₂—O—CH₃ | H | (RS) |
| 8.14 | Alb | F | CF₃ | 2-thienyl | H | (RS) |
| 9.1 | Alb | CF₃ | F | H | H | |
| 9.2 | Alb | CF₃ | F | CH₃ | H | (RS) |
| 9.3 | A5c-2) | CF₃ | F | CH₃ | H | (RS) |
| 9.4 | Alb | CF₃ | F | CH₂CH₃ | H | (RS) |
| 9.5 | A5c-2) | CF₃ | F | CH₂CH₃ | H | (S) |
| 9.6 | Alb | CF₃ | F | CH₂CH₂CH₃ | H | (RS) |
| 9.7 | A5c-2) | CF₃ | F | CH₂CH₂CH₃ | H | (S) |
| 9.8 | Alb | CF₃ | F | phenyl | H | (RS) |
| 9.9 | Alb | CF₃ | F | 3,4-dichlorophenyl | H | (RS) |
| 9.10 | Alb | CF₃ | F | cyclohexyl | H | (RS) |
| 9.11 | Alb | CF₃ | F | cyclopropyl | H | (RS) |
| 9.12 | Alb | CF₃ | F | CH₂—N(CH₃)₂ | H | (RS) |
| 9.13 | Alb | CF₃ | F | CH₂—O—CH₃ | H | (RS) |
| 9.14 | Alb | CF₃ | F | 2-thienyl | H | (RS) |

### B. Preparation of the Final Compounds

### Example B1

Preparation of Compound 1

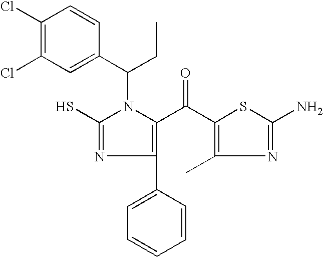

1N NaOH (0.006 mol) was added to a solution of intermediate 4 (prepared according to A2.a) (0.0026 mol) in ethanol, p.a. (10 ml), then the solution was stirred at 50° C. for 1 hour and was allowed to reach room temperature. The reaction mixture was poured out into H₂O (30 ml) and 1N HCl (7 ml) was slowly added. The resulting precipitate was filtered off, washed with H₂O and dried (vacuum) at 60° C. A part (0.68 g of 0.78 g) of the crude product was purified by high-performance liquid chromatography (eluent: (0.5% NH₄OAc in H₂O)/CH₃CN 90/10). The product fractions were collected and the organic solvent was evaporated. The aqueous concentrate was extracted with CH₂Cl₂, then the organic layer was separated, dried (MgSO₄) and filtered off. Finally, the solvent was evaporated. Yield: 0.24 g of compound 1.

### Example B2

a. Preparation of Compound 2

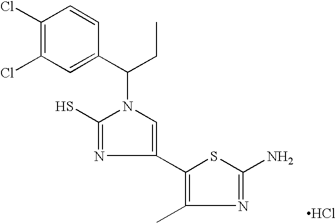

A solution of potassium thiocyanic acid salt (0.00285 mol) in H₂O (10 ml) was added to a stirring solution of intermediate S (prepared according to A2.b) (0.00095 mol) in methanol (10 ml), then 36% HCl, p.a. (0.00475 mol) was added and the reaction mixture was stirred for 4 hours at 55° C. Extra potassium thiocyanic acid salt (0.1 g) was added and the mixture was stirred for 18 hours at 55° C. The resulting mixture was allowed to reach room temperature and was left to stand for 1 hour. The formed precipitate was filtered off, washed with CH₃OH/H₂O (1/1) and dried (vacuum) at 60° C. Yield: 0.16 g of compound 2 (m.p.: 301.8-303.3° C.)
b-1. Preparation of Compound 3

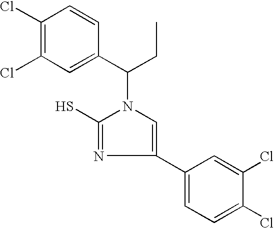

H₂O (2.5 ml), followed by potassium thiocyanic acid salt (0.00328 mol) and then concentrated HCl (0.5 ml) were added to a solution of intermediate 8 (prepared according to A3.c) (0.0013 mol) in methanol (15 ml) and the reaction mixture was stirred at room temperature for 20 hours, then the mixture was stirred at 55° C. for 48 hours and extra concentrated HCl (0.75 ml) was added. The resulting mixture was stirred at 60° C. for 24 hours, 1420 was added and the mixture was extracted with CH₂Cl₂. The organic layer was separated, dried (MgSO₄), filtered off and the solvent was evaporated. The residue was purified by reversed phase high-performance liquid chromatography (NH₄OAc 10% CH₃CN). The product fractions were collected and half of the solvent was evaporated. The resulting precipitate washed with H₂O and dried (vacuum) at 50° C. Yield: 0.061 g of compound 3.
b-2. Preparation of Compound 4

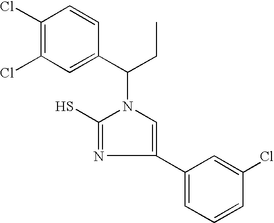

Potassium thiocyanic acid salt (0.000314 mol) and then HCl, 1N (0.00086 mol) were added to a solution of 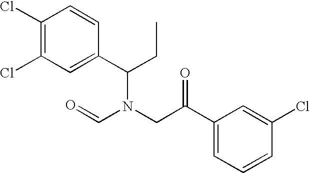
(prepared according to A3.c) (0.000286 mol) in methanol, p.a. (3 ml) and the reaction mixture was stirred at room temperature for 4 hours. HCl (0.5 ml, concentrated) was added and the mixture was stirred at 55° C. for 18 hours, then extra potassium thiocyanic acid salt (0.000515 mol) was added and the reaction mixture was stirred at 55° C. for 7 hours. The mixture was left to stand for 18 hours, H₂O (15 ml) was added and the product was extracted with CH₂Cl₂. The organic layer was separated and evaporated. The residue was purified by Fast high-performance liquid chromatography using the Reversed Phase-method. The product fractions were collected and the organic volatiles were evaporated. The product was extracted with CH₂Cl₂ and the separated organic layer was evaporated. Yield: 0.005 g of compound 4.

### Example B3

a. Preparation of Compound 5

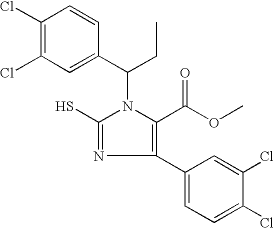

Intermediate 10 (prepared according to A4.b) (0.002 mol) was added at 0° C. to a mixture of N-(1-methylethyl)-2-propanamine lithium salt (0.0025 mol) in THF (40 ml) for 30 minutes and 3,4-dichlorobenzoyl chloride (0.0033 mol) was added, then the reaction mixture was reacted for 1 hour and quenched with water. The mixture was extracted with EtOAc, dried and the solvent was evaporated. The residue was diluted with CH₃OH and then concentrated HCl (q.s.) and potassium thiocyanic acid salt (1 g) were added. The resulting mixture was heated at 70° C. for 48 hours and partitioned between water and CH₂Cl₂. The organic layer was separated, dried and the solvent was evaporated. The residue was purified by high-performance liquid chromatography. The product fractions were collected and the solvent was evaporated. Yield: 0.180 g of compound 5.

b. Preparation of Compound 6

Method 1

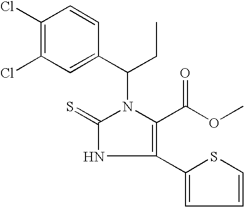

Intermediate 10 (prepared according to A4.b) (0.003 mol. in 10 ml THF) was added dropwise to a cold (−78° C.) solution of N-(1-methylethyl)-2-propanamine lithium salt (0.0036 mol. 2M in THF) in THF (5 ml). After 30 minutes 2-thiophenecarbonyl chloride (0.0031 mol) was added and the reaction mixture stood for 1 hour. The mixture was quenched with NH₄Cl and extracted with EtOAc. The organic layer was separated, dried (MgSO₄), filtered off and the solvent was evaporated. The residue was diluted with methanol (25 ml), then potassium thiocyanic acid salt (1 g) and concentrated HCl (1 ml) were added. The resulting mixture was heated overnight at 70° C. and the solvent was evaporated. The residue was partitioned between EtOAc and water, then the separated organic layer was dried and the solvent was evaporated. The residue was purified by high-performance liquid chromatography; the product fractions were collected and the solvent was evaporated. Yield: 0.040 g of compound 6.

c. Preparation of Compound 6

Method 2

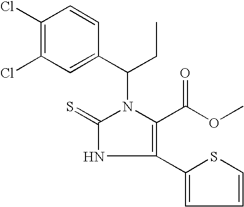

t-BuONa (0.150 g) was added at 0° C. to a mixture of intermediate 10 (prepared according to A4.b) (0.00082 mol) in THF (5 ml), then 2-thiophenecarbonyl chloride (0.001 mol) was added and after 30 minutes potassium thiocyanic acid salt (0.250 g) and concentrated HCl (1 ml) were added. The reaction mixture was heated at 70° C. for 18 hours and treated with water/CH₂Cl₂. The organic layer was separated, dried (MgSO₄), filtered off and the solvent was evaporated. The residue was purified by high-performance liquid chromatography, then the product fractions were collected and the solvent was evaporated. The residue was further purified by Flash column chromatography and then by normal phase high-performance liquid chromatography. The product fractions were collected and the solvent was evaporated. Yield: 45 mg of compound 6.
d-1. Preparation of Compound 7

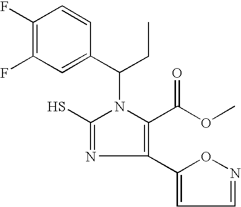

N-(1-methylethyl)-2-propanamine lithium salt (0.0184 mol. 2M in THF) was added dropwise at −78° C. under N₂ to a cold mixture of intermediate 29 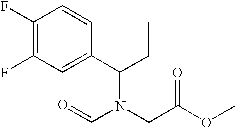
(prepared according to A4.b) (0.00924 mol) in THF (q.s.). After 30 minutes 5-isoxazolecarbonyl chloride (0.0110 mol) was added and then the reaction mixture was allowed to slowly reach room temperature. The mixture was quenched with NH₄Cl and the solvent was evaporated. The residue was diluted with CH₃OH, with H₂O and then potassium thiocyanic acid salt (3 g) and concentrated HCl were added. The reaction mixture was heated overnight at 80° C., then cooled, quenched with K₂CO₃ and extracted with CH₂Cl₂. The organic layer was separated, dried and the solvent was evaporated. The residue was purified by column chromatography over silica gel (gradient eluent: CH₂Cl₂/CH₃OH 100/0->90/10). Two product fractions were collected and the solvent was evaporated, to give Residue (I) and Residue (II). Residue (I) was purified by reversed phase high-performance liquid chromatography. The product fractions were collected and the solvent was evaporated. The residue was diluted with CH₂Cl₂ and washed 3 times with 1N HCl. The organic layer was separated, dried and the solvent was evaporated. Yield: 0.290 g of compound 7.
d-2. Preparation of Compound 43

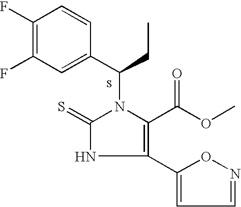

A solution of intermediate 34 (prepared according to A8.e) (0.0169 mol) in THF, p.a., dried on molecular sieves (80 ml) was stirred under N₂ on a 2-propanol/CO₂ cooling bath, then N-(1-methylethyl)-2-propanamine lithium salt (0.0179 mol; 2 M in THF/Heptane) was slowly added dropwise at −78° C. and the mixture was further stirred for 5 minutes. A solution of 5-isoxazolecarbonylchloride (0.02 mol) in THF, p.a., dried on molecular sieves (5 ml) was slowly added dropwise and the resulting mixture was stirred for 90 minutes at −78° C. and was then allowed to reach 10° C. A solution of KSCN (0.051 mol) in H₂O (30 ml) was added, followed by addition of CH₃OH (40 ml) and then concentrated HCl (14 ml). The reaction mixture was stirred for 4 hours at 60° C. and for 18 hours at 70° C., then the mixture was allowed to reach room temperature. The mixture was poured out into H₂O and was extracted with CH₂Cl₂. The organic layer was separated, dried (MgSO₄), filtered off and the solvent was evaporated. The residue was purified by high-performance liquid chromatography (Reversed Phase-method “NH₄OAc 10% CH₃CN—CH₃OH”). The product fractions were collected, then the organic solvent was evaporated and the aqueous concentrate was extracted with CH₂Cl₂. The organic layer was separated, dried (MgSO₄), filtered: off and the solvent was evaporated. Yield: 0.269 g of final compound 43.
d-3. Preparation of compound 53

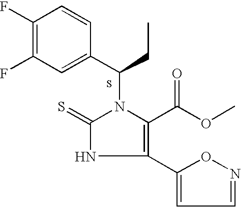

A mixture of intermediate 37 (prepared according to A9.c) (0.54 mol) in THF (1000 ml) was stirred and cooled to −78° C. on a CO₂/2-propanol-bath. 5-Isoxazolecarbonyl chloride (0.75 mol) was added. Then, 1,1,1-trimethyl-N-(trimethylsilyl)-silanamine lithium salt, 1M/THF (0.8 mol. 800 ml) was added over a 30 minutes period. The mixture was stirred for 4 hours at −78° C. Then, the reaction temperature was raised to −20° C. A solution of HCl, concentrated (66 ml) in water (400 ml) was added dropwise over 15 minutes. The organic solvent (THF) was evaporated. Water (400 ml) was added. Then, HCl, concentrated (280 ml) was added, followed by methanol (800 ml), then KSCN (0.8 mol). The reaction mixture was stirred for 22 hours at 60° C. Water was added. The mixture was cooled to room temperature, then treated with K₂CO₃ and NaHCO₃. This mixture was extracted with CH₂Cl₂. The separated organic layer was dried, filtered and the solvent evaporated. The residue (±200 g) was purified by high-performance liquid chromatography. The product fractions were collected and the solvent was evaporated. The residue was crystallized from DIPE (2×). The precipitate was filtered off and dried. Yield: 45.5 g of final compound 53 (mp: 128.5° C.).

### Example B4

Preparation of Compound 8

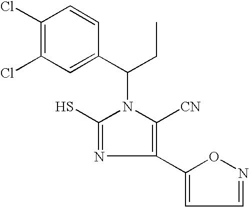

A solution of intermediate 15 (prepared according to A5.e) (0.00369 mol) in THF, p.a., dried on molecular sieves, (20 ml) was stirred on a 2-propanol/CO₂ cooling-bath and then N-(1-methylethyl)-2-propanamine lithium salt (0.0074 mol) was slowly added at −78° C. The resulting mixture was stirred for 15 minutes and 5-isoxazolecarbonyl chloride (0.0037 mol) was slowly added. The mixture was stirred for 10 minutes at −78° C. and was then allowed to reach room temperature. A solution of potassium thiocyanic acid salt (0.011 mol) in H₂O (15 ml) was added, followed by CH₃OH (10 ml) and then 36% HCl (1.3 ml). The reaction mixture was stirred for 4 hours at 60° C., poured out into ice-water and extracted with CH₂Cl₂/CH₃OH (98/2). The organic layer was separated, dried (MgSO₄), filtered off and the solvent was evaporated. The residue was purified by reversed phase high-performance liquid chromatography (eluent: (0.5% NH₄OAc in H₂O)/CH₃CN 10/90). The product fractions were collected and the organic solvent was evaporated. The aqueous residue was purified by Flash column chromatography on flash tubes (eluent: CH₂Cl₂/THF 92/8). The product fractions were collected, stirred in CH₂Cl₂/CH₃OH (98/2) and filtered to remove the silica Finally, the filtrate was evaporated. Yield: 0.020 g of compound 8.

### Example B5

Preparation of Compound 9

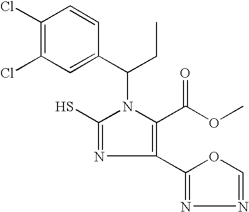

A solution of intermediate 19 (prepared according to A6.c) (0.00025 mol) in phosphorus oxychloride (5 ml) was stirred at 60° C. for 18 hours and then the solvent was evaporated. The residue was stirred in ice-water and extracted with CH₂Cl₂. The organic layer was separated, dried (MgSO₄), filtered off and the solvent was evaporated. The residue was stirred in 2-propanone (5 ml) and the mixture was treated with SO₂-gas for 15 minutes. The solvent was evaporated and the residue was purified by high-performance liquid chromatography. The product fractions were collected and the organic solvent was evaporated. The aqueous concentrate was extracted with CH₂Cl₂; the organic layer was separated, dried (MgSO₄), filtered off and the solvent was evaporated. Yield: 0.010 g of compound 9.

### Example B6

Preparation of Compound 10

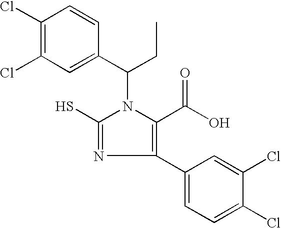

A mixture of compound 5 (prepared according to B3.a) (0.00025 mol) in 1N NaOH (1 ml) and methanol (3 ml) was reacted overnight at 70° C. and then the solvent was evaporated. The residue was treated with HCl and the mixture was filtered, then the desired product was dried (vacuum oven). Yield: 0.070 g of compound 10.

### Example B7

a. Preparation of Compound 11

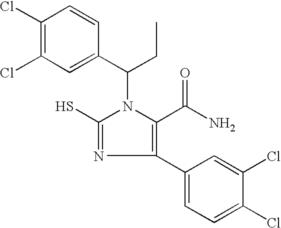

A mixture of compound 10 (prepared according to B6) (0.00015 mol) in SOCl₂ (5 ml) was heated at 70° C. for 4 hours, then the reaction mixture was cooled and the solvent was evaporated. The residue was diluted in THF and aqueous NH₄OH (3 ml) was added in one portion. The reaction mixture was stirred for 15 minutes and the solvent was evaporated. The residue was extracted with CH₂Cl₂ and then the organic layer was dried and the solvent was evaporated. The residue was taken up in 2-propanone and SO₂ (gas) was bubbled through the mixture for 10 minutes. The solvent was evaporated and the residue was purified by flash column chromatography (eluent: CH₂Cl₂/CH₃CN 95/5). The product fractions were collected and the solvent was evaporated. Yield: 15 mg of compound 11.
b. Preparation of Compound 13

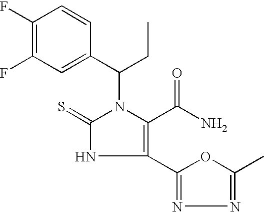

A mixture of compound 12 (prepared according to B6) (0.0012 mol), N′-(ethylcarbonimidoyl)-N,N-dimethyl-1,3-propanediamine (0.0013 mol) and 1-hydroxy-1H-benzotriazole (0.0013 mol) in DMF (8 ml) was stirred for 30 minutes at room temperature, then NH₃ (gas) was passed through the solution for 15 minutes and the reaction mixture was stirred for 1 hour. The solvent was evaporated and the obtained residue was stirred in H₂O, then the mixture was extracted with CH₂Cl₂. The organic layer was separated, dried, filtered off and the solvent was evaporated. The residue was purified by column chromatography over silica gel (eluent: CH₂Cl₂/CH₃OH 96/4). The pure product fractions were collected and the solvent was evaporated. The residue was stirred in DIPE and the resulting solids were collected. Yield: 0.190 g. This fraction was purified by reversed-phase high-performance liquid chromatography, then the pure product fractions were collected and the solvent was evaporated. The obtained residue was stirred in DIPE and then the desired product was filtered off and dried. Yield: 0.091 g of compound 13 (m.p.: 228-229° C.).

### Example B8

Preparation of Compound 14

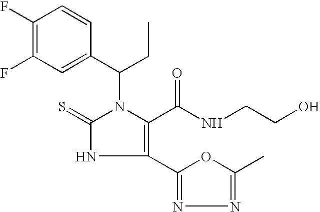

A mixture of compound 12 (prepared according to B6) (0.0012 mol), N′-(ethylcarbonimidoyl)-ANN-dimethyl-1,3-propanediamine (0.0012 mol) and 1-hydroxy-1H-benzotriazole (0.0012 mol) in DMF (8 ml) was stirred for 30 minutes at room temperature, then 2-aminoethanol (0.0024 mol) was added and the reaction mixture was stirred for 3 hours at room temperature. The solvent was evaporated and the obtained residue was purified by column chromatography over silica gel (eluent: CH₂Cl₂/CH₃OH 96/4). The pure product fractions were collected and the solvent was evaporated. The residue was stirred in DIPE and then the resulting solids were filtered off and dried. Yield: 0.155 g of compound 14 (m.p.: 165-166° C.).

### Example B9

Preparation of Compound 16

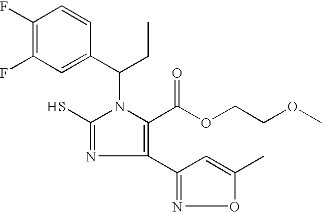

A mixture of compound 15 (prepared according to 133) (0.00029 mol) and NaBH₄ (0.00211 mol) in 2-methoxyethanol (5 ml) was heated for 24 hours at 100° C. and then the solvent was evaporated. The residue was purified by reversed-phase high-performance liquid chromatography. The product fractions were collected and the solvent was evaporated Yield: 0.020 g of compound 16.

### Example B110

Preparation of Compound 50

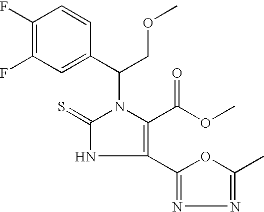

A mixture of intermediate 26 (prepared according to A7.c) (0.00125 mol) and Burgess' reagent (0.00375 mol) in THF (10 ml) was stirred for 3 hours at room temperature, then CH₂Cl₂ was added and the mixture washed with H₂O. The organic layer was separated, dried, filtered off and the solvent was evaporated. The obtained residue was filtered over silica gel (eluent: CH₂Cl₂/CH₃OH 98/2), then the product fractions were collected and the solvent was evaporated. The residue was stirred in DIPE and the desired product was filtered off and dried. Yield: 0.082 g of final compound 50 (m.p.: 102.8-102.9° C.).

Table 2 and 3 list the compounds of formula (I) which were prepared according to one of the above samples (Ex. No.)

TABLE 2

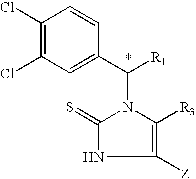

| Comp. | Exp. | | | | |
|---|---|---|---|---|---|
| No. | No. | R1 | R3 | Z | Properties |
| 17 | B2.b-1 | —CH₂CH₃ | H | 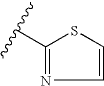 | HCl (1:1) |
| 2 | B2.a | —CH₂CH₃ | H | 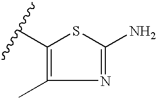 | HCl (1:1) |
| 18 | B2.a | —CH₂CH₃ | H | 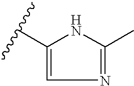 | |
| 4 | B2.b-2 | —CH₂CH₃ | H | 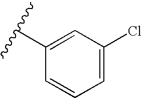 | |
| 3 | B2.b-1 | —CH₂CH₃ | H | 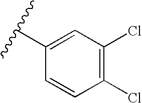 | |
| 19 | B2.b-1 | —CH₂CH₃ | H | 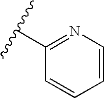 | HCl(1:1) |
| 20 | B2.a | —CH₂CH₃ | H | 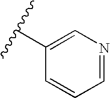 | HCl (1:1) |
| 21 | B4 | —CH₂CH₃ | 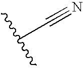 | 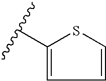 | |
| 8 | B4 | —CH₂CH₃ | 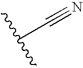 | 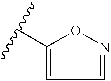 | |
| 38 | B4 | —CH₂CH₃ | 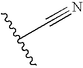 | 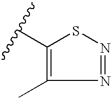 | |
| 39 | B6 | —CH₂CH₃ | 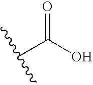 | 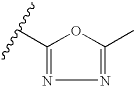 | |
| 22 | B6 | —CH₂CH₃ | 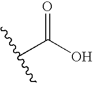 | 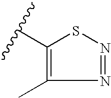 | |
| 10 | B6 | —CH₂CH₃ | 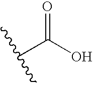 | 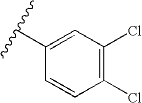 | |
| 23 | B6 | —CH₂CH₃ | 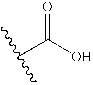 | 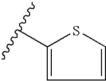 | |
| 6 | B3.b/c | —CH₂CH₃ | 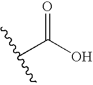 | 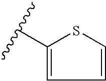 | |
| 24 | B3.c | —CH₂CH₃ | 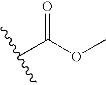 | 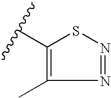 | |
| 25 | B3.abc | —CH₂CH₃ | 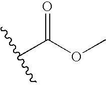 | 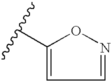 | |
| 26 | B3.abc | —CH₂CH₃ | 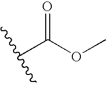 | 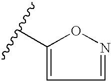 | m.p. 165-166° C.; *(S) |
| 9 | B5 | —CH₂CH₃ | 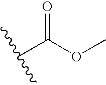 | 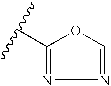 | |
| 27 | B5 | —CH₂CH₃ | 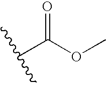 | 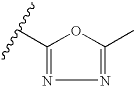 | |
| 28 | B3.abc | —CH₂CH₃ | 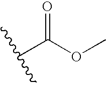 | 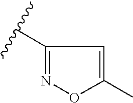 | m.p. 163-164° C. |
| 29 | B3.abc | —CH₂CH₃ | 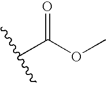 | 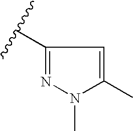 | m.p. 107.8- 108.3° C. |
| 30 | B3.abc | —CH₂CH₃ | 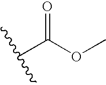 | 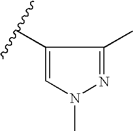 | m.p. 147.9- 149.3° C. |
| 31 | B3.abc | —CH₂CH₃ | 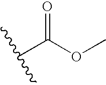 | 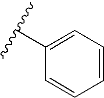 | |
| 32 | B3.abc | —CH₂CH₃ | 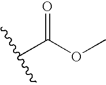 | 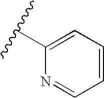 | m.p. 187.6-189.1° C. |
| 33 | B3.abc | —CH₂CH₃ | 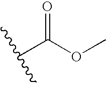 | 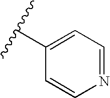 | m.p. 168.9-171.5° C. |
| 34 | B3.abc | —CH₂CH₃ | 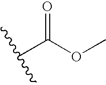 | 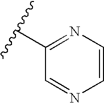 | m.p. 147.9-149.3° C. |
| 5 | B3.a | —CH₂CH₃ | 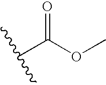 | 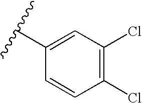 | |
| 35 | B3.abc | —CH₂CH₃ | 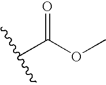 | 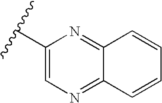 | |
| 36 | B7.b | —CH₂CH₃ | 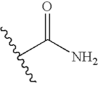 | 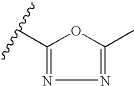 | m.p. 236-237° C. |
| 11 | B7.a | —CH₂CH₃ | 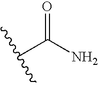 | 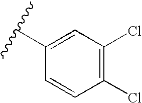 | |
| 37 | B8 | —CH₂CH₃ | 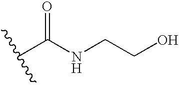 | 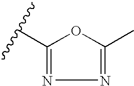 | m.p. 204-206° C. |
| 1 | B1 | —CH₂CH₃ | 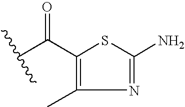 | 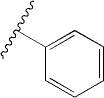 | |
| 40 | B5 | —CH₂CH₂CH₃ | 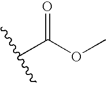 | 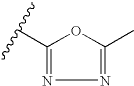 | |
| 51 | B3abc | —CH₂CH₃ | 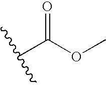 | 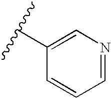 | •HCl (1:1) |
| 52 | B5 | —CH₂CH₃ | 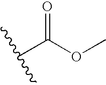 | 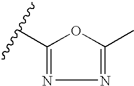 | * (S) m.p. 154.2- 154.4° C. |

TABLE 3

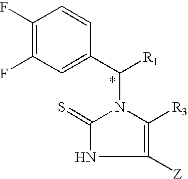

| Comp. | | | | | |
|---|---|---|---|---|---|
| No. | Exp. No. | R1 | R3 | Z | Properties |
| 41 | B3.abc | —CH₃ | 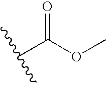 | 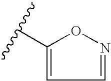 | |
| 42 | B5 | —CH₃ | 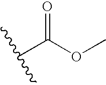 | 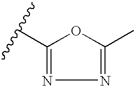 | m.p. 192-194° C. |
| 12 | B6 | —CH₂CH₃ | 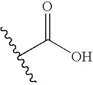 | 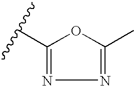 | |
| 7 | B3.d-1 | —CH₂CH₃ | 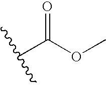 | 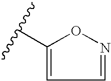 | |
| 43 | B3.d-2 | —CH₂CH₃ | 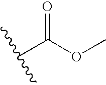 | 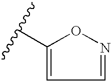 | * (S); rot¹.- 140.6°; m.p. 111.5° C. |
| 15 | B3 | —CH₂CH₃ | 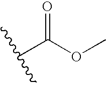 | 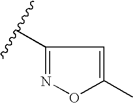 | |
| 44 | B5 | —CH₂CH₃ | 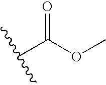 | 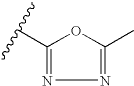 | |
| 45 | B3.abc | —CH₂CH₃ | 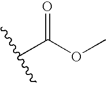 | 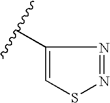 | |
| 46 | B3.abc | —CH₂CH₃ | 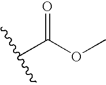 | 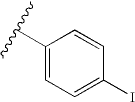 | |
| 47 | B7.b | —CH₂CH₃ | 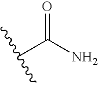 | 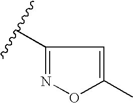 | |
| 13 | B7.b | —CH₂CH₃ | 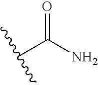 | 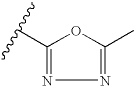 | m.p. 228-229° C. |
| 14 | B8 | —CH₂CH₃ | 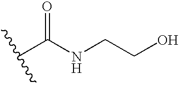 | 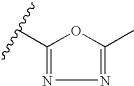 | m.p. 165-166° C. |
| 16 | B9 | —CH₂CH₃ | 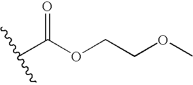 | 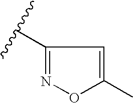 | |
| 48 | B3.abc | —CH₂CH₂CH₃ | 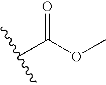 | 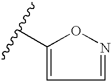 | |
| 49 | B3.abc | —CH₂CH₂CH₃ | 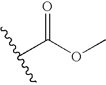 | 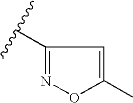 | m.p. 141° C. |
| 50 | B10 | —CH₂OCH₃ | 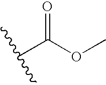 | 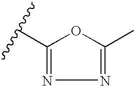 | m.p. 102.8- 102.9° C. |
| 53 | B3.d-3 | —CH₂CH₃ | 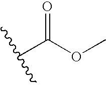 | 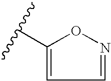 | * (S); rot². -172°; m.p. 128.5° C. |

¹[α]₂₀^{D} at concentration of 0.030 g/100 ml in CHCl₃;
²[α]₂₀^{D} at concentration of 0.5 g/100 g in CHCl₃;

### C. Analytical Part

LCMS Conditions 1

The HPLC gradient was supplied by a Waters Alliance HT 2790 system with a column heater set at 40° C. Flow from the column was split to a Waters 996 photodiode array (PDA) detector and a Waters-Micromass ZQ mass spectrometer with an electrospray ionization source operated in positive and negative ionization mode. Reversed phase HPLC was carried out on a Xterra MS C18 column (3.5 μm, 4.6×100 mm) (12 minutes column) with a flow rate of 1.6 ml/minutes. Three mobile phases (mobile phase A: 95% 25 mM ammoniumacetate+5% acetonitrile; mobile phase B: acetonitrile; mobile phase C: methanol) were employed to run a gradient condition from 100% A to 50% B and 50% C in 6.5 minutes, to 100% B in 1 minute, 100% B for 1 minute and reequilibrate with 100% A for 1.5 minute. An injection volume of 10 μL was used.

Mass spectra were acquired by scanning from 100 to 1000 in Is using a dwell time of 0.1 s. The capillary needle voltage was 3 kV and the source temperature was maintained at 140° C. Nitrogen was used as the nebulizer gas. Cone voltage was 10 V for positive ionization mode and 20 V for negative ionization mode. Data acquisition was performed with a Waters-Micromass MassLynx-Openlynx data system.

LCMS Conditions 2 Speed Analysis

The HPLC gradient was supplied by a Waters Alliance 2690 system with a column heater set at 50° C. Flow from the column was split to a Waters 996 photodiode array (PDA) detector and a Waters-Micromass ZQ mass spectrometer with an electrospray ionization source operated in positive and negative ionization mode. Reversed phase HPLC was carried out on a Xterra MS C18 column (2.5 μm, 4.6×20 mm) with a flow rate of 3 ml/min. Three mobile phases (mobile phase A 95% 25 mM anmuoniumacetate+5% acetonitrile; mobile phase B: acetonitrile; mobile phase C: methanol) were employed to run a gradient condition from 100% A to 50% B and 50% C in 0.9 min., to 100% B in 0.37 min, 100% B for 0.18 min. and reequilibrate with 100% A for 0.2 min. An injection volume of 2 μL was used.

Mass spectra were acquired by scanning from 100 to 1000 in is using a dwell time of 0.1 s. The capillary needle voltage was 3 kV and the source temperature was maintained at 140° C. Nitrogen was used a the nebulizer gas. Cone voltage was 10 V for positive ionization mode and 20 V for negative ionization mode. Data acquisition was performed with a Waters-Micromass MassLynx-Openlynx data system.

TABLE 4
LCMS parent peak ([M⁺] defines the mass
of the compound) and retention time (minutes)

| | | | LCMS |
|---|---|---|---|
| Compound no. | [MH+] | Retention time | condition |
| 31 | 421 | 6.24 | 1 |
| 6 | 427 | 6.24 | 1 |
| 27 | 427 | 5.02 | 1 |
| 5 | 488 | 6.35 | 1 |
| 9 | 413 | 4.82 | 1 |
| 11 | 473 | 5.45 | 1 |
| 4 | 397 | 6.54 | 1 |
| 19 | 364* | 5.54 | 1 |
| 17 | 370* | 6.20 | 1 |
| 3 | 433 | 4.76 | 1 |
| 24 | 443 | 5.34 | 1 |
| 40 | 441 | 5.12 | 1 |
| 35 | 473 | 5.91 | 1 |
| 1 | 503 | 5.65 | 1 |
| 20 | 364* | 4.57 | 1 |
| 18 | 367 | 5.24 | 1 |
| 25 | 412 | 5.60 | 1 |
| 2 | 399* | 5.51 | 1 |
| 21 | 394 | 4.81 | 1 |
| 10 | 475 | 4.40 | 1 |
| 8 | 377(−) | 5.34 | 1 |
| 7 | 378 | 5.59 | 1 |
| 38 | 410 | 4.95 | 1 |
| 15 | 393 | 5.61 | 1 |
| 45 | 396 | 5.74 | 1 |
| 47 | 378 | 3.57 | 1 |
| 46 | 514 | 6.62 | 1 |
| 16 | 437 | 5.33 | 1 |
| 51 | 422 | 1.02 | 2 |

[M⁺] defines the mass of the compound
*mass of the corresponding base

### D. Pharmacological Example

Inhibition of MCP-1 Induced Ca-Flux in Human THP-1 Cells

MCP-1 binding to the CCR2 receptor induces a rapid and transient intracellular release of Ca²⁺ (secondary messenger) in several cell lines (Charo et al, PNAS 1994). Free Ca²⁺ levels can be measured using a Ca²⁺ sensitive dye. When the CCR2 receptor is blocked with a CCR2 receptor antagonist, the MCP-1 induced release of Ca²⁺ is inhibited.

Human THP-1 cells (monocytic cell line, ATCC TIB-202) were cultured in RPMI 1640 medium supplemented with 10% fetal calf serum (FCS), 1% L-Glutamine, penicillin (50 U/ml) and streptomycin (50 μg/ml) (all GIBCO BRL, Gent). After centrifugation, cells were loaded for 30 minutes with the Ca²⁺ sensitive fluorescent dye Fluo-3 μM (Molecular Probes, Leiden, Netherlands) (2 million cells/ml in RPMI medium containing 4 μM Fluo-3 μM, 20 mM HEPES, 0.1% Bovine Serum Albumin (BSA) and 5 mM probenecid). Excess dye was removed by 3-fold washing with buffer (5 mM HEPES, 140 mM NaCl, 1 mM MgCl₂, 5 mM KCl, 10 mM glucose, 2.5 mM probenecid, 1.25 mM CaCl₂, 0.1% BSA; all further incubations were done in this buffer). Cells were plated at a density of 150 000 cells/well in dark-wall 96-well plates (Costar, Cambridge, Mass.) and sedimented by centrifugation (1 minute). The cells were pre-incubated for 20 minutes with test compound. Then, 10⁻⁷ M hMCP-1 (Bachem, Bubendorf, Switserland) was added. Changes in intracellular free Ca²⁺ concentration were measured using the Fluorescent Imaging Plate Reader (FLIPR, Molecular Devices, Munchen, Germany). Fluorescence was recorded every second from 10 seconds before the addition of the MCP-1 till 2 minutes after the addition (first minute: 60 records with 1 second intervals, second minute 20 records with 3 second intervals). The maximal fluorescence obtained during this time frame was used for further calculations.

Table 5 reports pIC₅₀ values obtained in the above-described test for compounds of formula (I). pIC₅₀ defines—log IC₅₀ wherein IC₅₀ is the molar concentration of the test compound which inhibits 50% of specific MCP-1 induced Ca²⁺ flux.

TABLE 5

| Comp. No. | pIC₅₀ |
|---|---|
| 31 | 7.12 |
| 24 | 7.14 |
| 6 | 7.26 |
| 40 | 7.36 |
| 36 | 7.4 |
| 51 | 7.4 |
| 9 | 7.45 |
| 27 | 7.48 |
| 45 | 7.5 |
| 16 | 7.5 |
| 49 | 7.6 |
| 32 | 7.6 |
| 34 | 7.6 |
| 28 | 7.8 |
| 25 | 7.88 |
| 48 | 7.9 |
| 33 | 7.9 |
| 7 | 7.93 |
| 26 | 8.1 |
| 43/53 | 8.1 |

Radioligand Binding Assay.

¹²⁵I-MCP-1 binding assays were performed in 96:well plates with 40 μg of protein per well. Compounds were dissolved and diluted in DMSO to 100× dilutions. A 10× concentration range of compounds was prepared in binding buffer (10% DSMO). Competition binding assays contained the following components in a total volume of 250 μl: 25 μl of the appropriate compound dilution (final concentration of 1% DMSO), 200 μl membranes from CCR2B-transfected CHO cells dissolved in binding buffer and 25 μl ¹²⁵I-MCP-1 (Bolton and Hunter labeled, Amersham, specific activity=2000 Ci/mmol, 0.15 nM final). Binding buffer was composed of 25 mM HEPES, 5 mM MgCl₂, 1 mM CaCl₂, 0.5% protease-free bovine serum albumin, pH 7.4. After 90 minutes incubation at 25° C. membranes were harvested on GF/B filters—presoaked in 0.5% polyethylenimine, followed by washing with buffer containing 25 mM HEPES, 5 mM MgCl₂, 1 mM CaCl₂, 5 mM NaCl, pH 7.4. Filter bound radioactivity was determined by liquid scintillation counting. EC₅₀ values (μM) and Kᵢ values (μM) were calculated. The ED₅₀ value indicates the concentration of the test compound that competes with MCP-1 for half of the specific binding sites; the Kᵢ value indicates the equilibrium dissociation constant, i.e. the concentration of the test compound that will bind to half of the binding sites at equilibrium in the absence of radioligand or other competitors. EC₅₀ values and Kᵢ values were calculated using non-linear regression in Graphpad Prism. Prism calculates the Kᵢ or affinity of the receptor for the competing drug using the equation of Cheng and Prusoff (Biochem. Pharmacol. 1973, 22: 3099-3108). A low Kᵢ indicates a high affinity of the receptor for the test compound.

$${K}_{i} = \frac{{EC}_{50}}{1 + \frac{[ radioligand ]}{{K}_{d}}}$$

wherein K_{d} describes the affinity of the radioligand for the receptor, i.e. the concentration of the radioligand that will bind to half of the binding sites at equilibrium in the absence of competitors.

Table 6 lists Kᵢ values (μM) obtained in the above-described test for compounds of formula (I).

TABLE 6

| Comp. No. | Kᵢ values |
|---|---|
| 13 | 0.30 |
| 15 | 0.35 |
| 26 | 0.05 |
| 28 | 0.01 |
| 31 | 0.07 |
| 36 | 0.07 |
| 43/53 | 0.03 |

Chemotactic Response

The CCR2 antagonistic activity of the compounds of the present invention can also be determined by measuring the effect of the compounds on the chemotactic response of cells in the presence of a chemokine, such as for example MCP-1.

Mononuclear cells from human heparinized peripheral blood (PBMC) were isolated using Ficoll-Paque gradient centrifugation (Amersham Biosciences). Assays of chemotactic responsiveness were performed using disposable 96-well chemotaxis chambers (ChemoTx, Neuro Probe) with 5-μm pore size polycarbonate (PVP-free) filter membranes. Mononuclear cells were fluorescently labeled with 5 μg/ml Calcein-AM (Molecular Probes, Eugene, Oreg.) at 37° C. for 30 minutes. Labeled cells were washed twice and resuspended at 5×10⁶ cells/ml in Hanks' Balanced Salt Solution (Gibco BRL) supplemented with 0.2% bovine serum albumin. Subsequently, cells were pre-incubated for 10 minutes at room temperature with serial dilutions of the compounds in DMSO (dimethylsulfoxide) (final DMSO concentration of 0.2%). Bottom wells of the chemotaxis chamber were loaded with 28 μl medium containing 30 ng/ml recombinant hMCP-1 (R&D) or buffer only. Pre-treated cells (100.000 cells) were added in triplicate to the topside of the filter (20 μl) and incubated at 37° C. in humidified air containing 5% CO₂. After 105 minutes incubation, the non-migrated cells were removed from the top of the filter by gently wiping the filter with a tissue. The migrated cells were measured using a fluorescent plate reader (λ_{excitation}=485 nm; λₑₘᵢₛₛᵢₒₙ=538 nm). The chemotactic response can be expressed as chemotactic index (C.I.), being the ratio of the means of migrated cells in the presence of MCP-1 and the means of migrated cells in the absence of chemokine. Percentage inhibition was calculated using the formula:

$$\% inhibition = ( 1 - \frac{{F}_{sample} - {F}_{buffer}}{{F}_{MCP - 1} - {F}_{buffer}} ) \times 100$$

with Fₛₐₘₚₗₑ, the fluorescence of the cells pre-incubated with 10, 1, 0.1, 0.01 or 0.001 μM compound and migrated to 30 ng/ml MCP-1 in the bottom wells; F_{MCP-1}, the fluorescence of the cells pre-incubated with buffer-0.2% DMSO and migrated to 30 ng/ml MCP-1 and F_{buffer}, the fluorescence of cells pre-incubated with buffer-0.2% DMSO and spontaneous migrated to buffer in the bottom wells. Table 7 lists the IC₅₀ values (μM) obtained in the above-described test for compounds of formula (I). Assays were run in triplicate and repeated 2-7 times.

TABLE 7

| Compound no. | IC50 (μM) |
|---|---|
| 23 | 0.024 |
| 53 | 0.108 |
| 31 | 0.214 |
| 36 | 0.244 |
| 28 | 0.367 |
| 51 | 0.474 |
| 15 | 0.798 |

1. A compound of formula 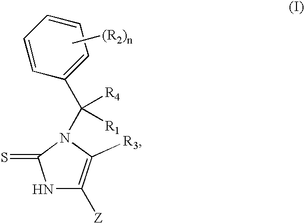
a N-oxide, a pharmaceutically acceptable addition salt, a quaternary amine, a polymorphic form or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or
C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₆alkyl, mono- or di(C₁₋₄alkyl)aminoC₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 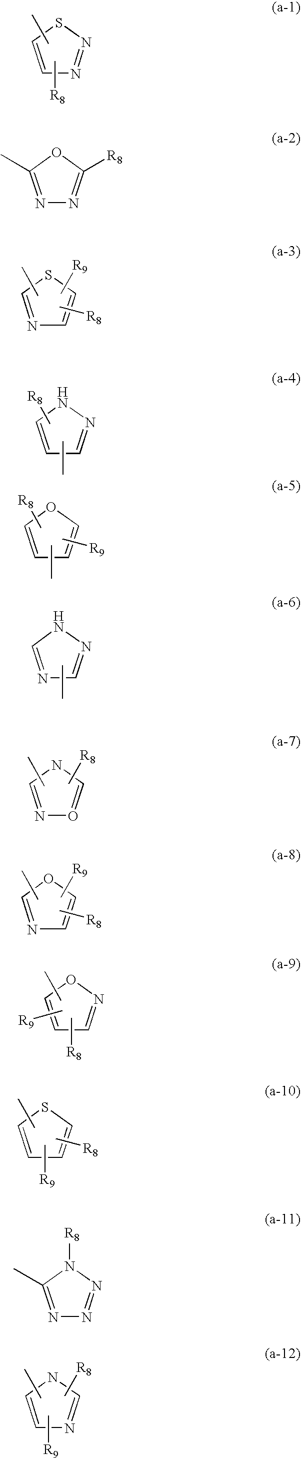 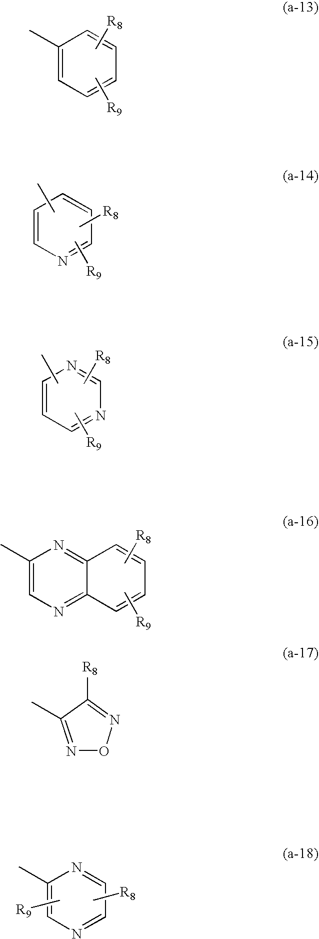
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo,
C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro;
provided that
1-(3,4-dimethoxybenzyl)-4-phenyl-1H-imidazole-2-thiol; and
1-(o-chlorobenzyl)-5-ethyl-4-phenyl-imidazole-2-thiol
are not included.

2. A compound according to claim 1 wherein R₂ represents halo, C₁₋₆alkyl, C₁₋₆alkyloxy or polyhaloC₁₋₆alkyl.

3. A compound according to claim 2 wherein R₂ represents halo or polyhaloC₁₋₆alkyl.

4. A compound according to claim 3 wherein R₂ represents halo.

5. A compound according to claim 4 wherein R₂ represents fluoro.

6. A compound according to claim 4 wherein n is 2 or 3.

7. A compound according to claim 6 wherein n is 2.

8. A compound according to claim 7 wherein n is 2 and said two R² substituents are placed in meta and para postion.

9. A compound according to claim 1 wherein Z represents a radical of formula (a-1), (a-2), (a-3), (a-9), (a-10), (a-12), (a-13), (a-14) or (a-16).

10. A compound according to claim 9 wherein Z represents a radical of formula (a-1), (a-2), (a-9), (a-10) or (a-13).

11. A compound according to claim 10 wherein Z represents a radical of formula (a-9).

12. A compound according to claim 1 wherein R₃ represents hydrogen, cyano, C(═O)—O—R₅, C(═O)—NR₆ₐR_{6b}, C(═S)—NR₆ₐR₆b, S(═O)₂—NR₆ₐR_{6b} or C(═O)—R₇.

13. A compound according to claim 12 wherein R₃ represents cyano, C(═O)—O—R₅, C(═O)—NR₆ₐR_{6b}, C(═S)—NR₆ₐR_{6b}, S(═O)₂—NR₆ₐR_{6b} or C(═O)—R₇.

14. A compound according to claim 13 wherein R₃ represents C(═O)—O—R₅.

15. A compound according to claim 14 wherein R₃ represents methoxycarbonyl.

16. A compound according to claim 1 wherein R₁ represents C₁₋₆alkyl or C₁₋₆alkyloxyalkyl.

17. A compound according to claim 16 wherein R₁ represents C₁₋₆alkyl.

18. A compound according to claim 17 wherein R₁ represents ethyl.

19. A compound according to claim 1 wherein R₄ represents hydrogen.

20. A compound according to claim 1 wherein R₁ represents C₁₋₆alkyl or C₁₋₆alkyloxyalkyl; R₂ represents halo; R₃ represents hydrogen, cyano, C(═O)—O—R₅, C(═O)—NR₆ₐR_{6b}, C(═O)—R₇; Z represents a ring system selected from (a-1), (a-2), (a-3), (a-9), (a-10), (a-12), (a-13), (a-14) or (a-16); R₄ represents hydrogen; n is 2.

21. A compound according to claim 1 wherein the compound is stereochemically pure.

22. A compound according to claim 1 wherein the compound has the following formula

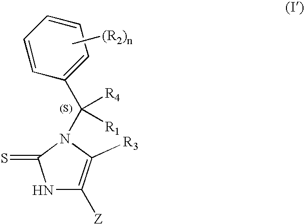

23. A compound according to claim 1 wherein the compound has the following formula

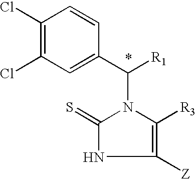

| | | | |
|---|---|---|---|
| —CH₂CH₃ | 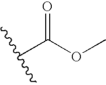 | 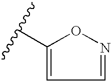 | *(S) |
| —CH₂CH₃ | 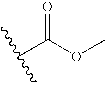 | 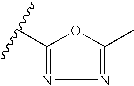 | *(S) |

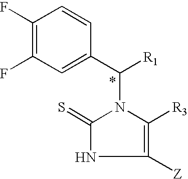
| | | | |
|---|---|---|---|
| —CH₂CH₂CH₃ | 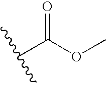 | 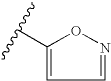 | |
| —CH₂CH₃ | 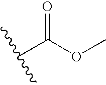 | 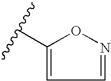 | * (S); m.p. 111.5° C. |
| —CH₂CH₃ | 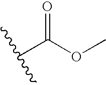 | 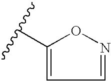 | * (S); m.p. 128.5° C. |

24. A compound according to claim 1 wherein the compound is (S)-3-[1-(3,4-difluoro-phenyl)-propyl]-5-isoxazol-5-yl-2-thioxo-2,3-dihydro-1H-imidazole-4-carboxylic acid methyl ester, a N-oxide, a pharmaceutically acceptable addition salt, a quaternary amine or a polymorphic form thereof.

25. A compound according to claim 24 wherein the compound is (S)-3-[1-(3,4-difluoro-phenyl)-propyl]-5-isoxazol-5-yl-2-thioxo-2,3-dihydro-1H-imidazole-4-carboxylic acid methyl ester or a N-oxide thereof.

26. A compound according to claim 24 wherein the compound is (S)-3-[1-(3,4-difluoro-phenyl)-propyl]-5-isoxazol-5-yl-2-thioxo-2,3-dihydro-1H-imidazole-4-carboxylic acid methyl ester or a pharmaceutically acceptable addition salt thereof.

27. A compound according to claim 24 wherein the compound is (S)-3-[1-(3,4-difluoro-phenyl)-propyl]-5-isoxazol-5-yl-2-thioxo-2,3-dihydro-1H-imidazole-4-carboxylic acid methyl ester or a quaternary amine thereof.

28. A compound according to claim 24 wherein the compound is (S)-3-[1-(3,4-difluoro-phenyl)-propyl]-5-isoxazol-5-yl-2-thioxo-2,3-dihydro-1H-imidazole-4-carboxylic acid methyl ester.

29. A compound according to claim 24 wherein the compound is (S)-3-[1-(3,4-difluoro-phenyl)-propyl]-5-isoxazol-5-yl-2-thioxo-2,3-dihydro-1H-imidazole-4-carboxylic acid methyl ester with a melting point of 128.5° C.

30. (canceled)

31. A method for preventing or treating diseases mediated through activation of the CCR2 receptor comprising administering to a subject in need thereof a therapeutically effective amount of a compound of formula (I) 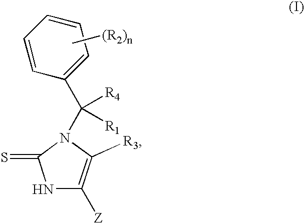
a N-oxide, a pharmaceutically acceptable addition salt, a quaternary amine, a polymorphic form or a stereochemically isomeric form thereof, wherein
R₁ represents hydrogen, C₁₋₆alkyl, C₃₋₇cycloalkyl, C₁₋₆alkyloxyC₁₋₆alkyl, di(C₁₋₆alkyl)aminoC₁₋₆alkyl, aryl or heteroaryl;
each R₂ independently represents halo, C₁₋₆alkyl, C₁₋₆alkyloxy, C₁₋₆alkylthio, polyhaloC₁₋₆alkyl, polyhaloC₁₋₆alkyloxy, cyano, aminocarbonyl, amino, mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)—NR₆ₐR_{6b}, S(═O)₂—NR₆ₐR_{6b} or C(═O)—R₇;
R₄ represents hydrogen or C₁₋₆alkyl;
R₅ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl or aryl;
R₆ₐ and R_{6b} each independently represent hydrogen, C₁₋₆alkyl, amino, mono- or di(C₁₋₄alkyl)amino, arylNH—, aminoC₁₋₁₆alkyl, mono- or di(C₁₋₄alkyl)amino C₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl, imidazolidinyl, pyrazolidinyl, piperidinyl, piperazinyl, morpholinyl, thiomorpholinyl or piperazinyl substituted with C₁₋₆alkyl;
R₇ represents hydrogen, C₁₋₆alkyl, hydroxyC₁₋₆alkyl, C₂₋₆alkenyl, C₂₋₆alkynyl, polyhaloC₁₋₆alkyl, C₁₋₆alkyloxyC₁₋₆alkyl, aminoC₁₋₆alkyl, mono- or di(C₁₋₄alkyl)aminoC₁₋₆alkyl, aminocarbonylC₁₋₆alkyl, mono- or di(C₁₋₄alkyl)aminocarbonylC₁₋₆alkyl, aryl or heteroaryl;
Z represents a cyclic ring system selected from 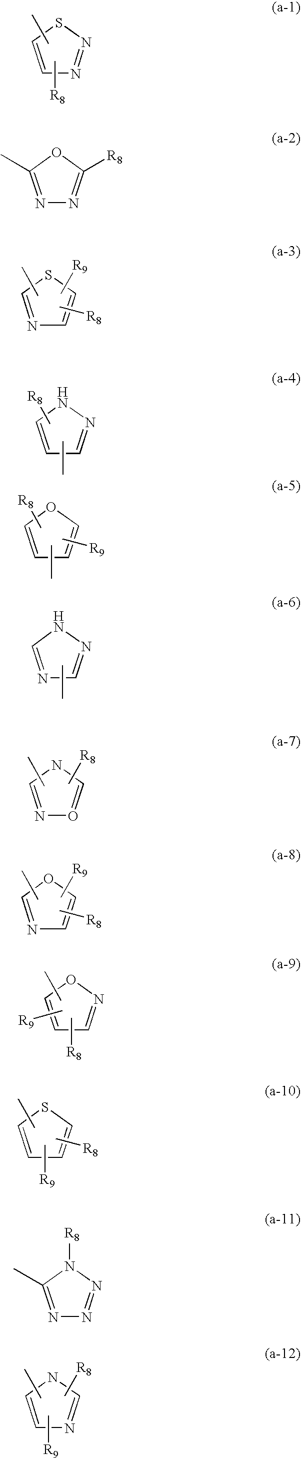 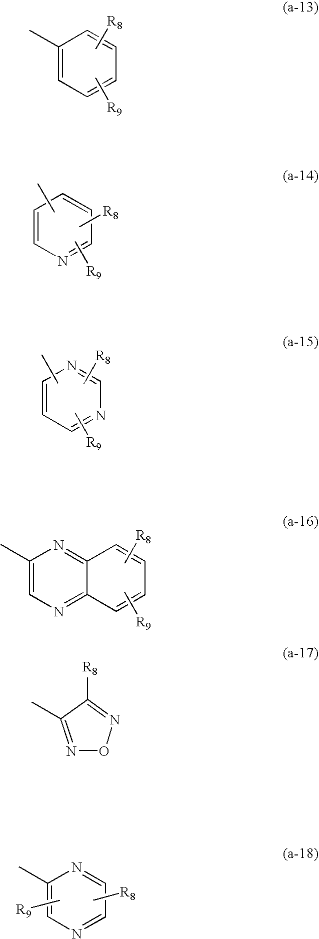
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, hydroxyC₁₋₆alkylamino, aryl, aryloxy, piperidinyl, piperidinylamino, morpholinyl, piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, each of said heterocycles optionally being substituted with one or two substituents each independently selected from halo,
C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl, amino, mono- or di(C₁₋₄alkyl)amino or nitro.

32. (canceled)

33. The method according to claim 31 wherein the disease is an inflammatory disease.

34. A pharmaceutical composition comprising a pharmaceutically acceptable carrier, and as active ingredient a therapeutically effective amount of a compound as claimed in claim 1.

35. A process of preparing a pharmaceutical composition comprising mixing a pharmaceutically acceptable carrier with a therapeutically effective amount of a compound as claimed in claim 1.

36. A process of preparing a compound of formula (I)
a) by reacting an intermediate of formula (II-a) or (II-b) with KSCN in the presence of a suitable acid and a suitable solvent, 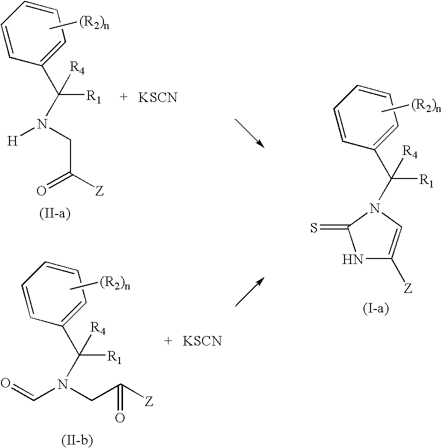
b) reacting an intermediate of formula (III) with an intermediate of formula (IV) wherein W₁ represents a suitable leaving group, in the presence of KSCN, a suitable acid, a suitable solvent, and a suitable base, 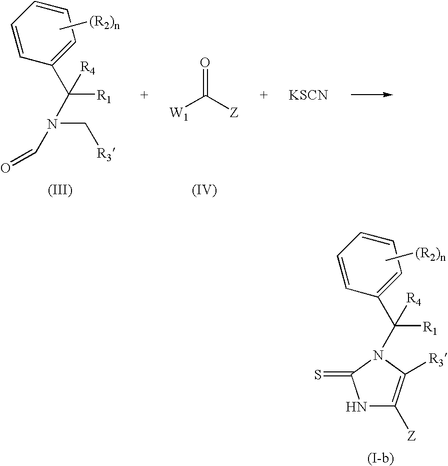
with R₃ representing R₃ other than hydrogen;
c) reacting an intermediate of formula (V) with a suitable base in the presence of a suitable solvent 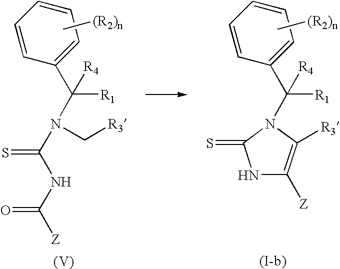
with R₃ representing R₃ other than hydrogen;
d) reacting an intermediate of formula (VI) with phosphoric trichloride or Burgess' reagent optionally in the presence of a suitable solvent 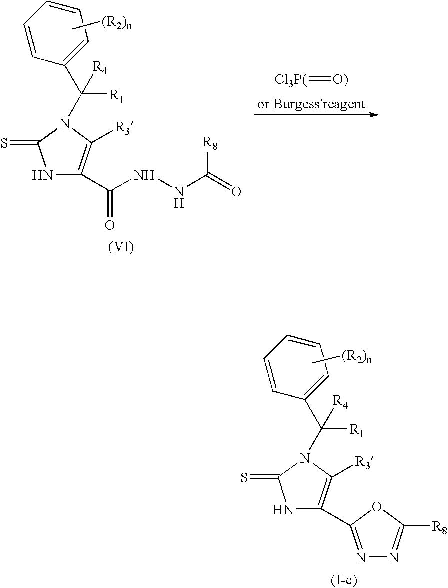
e) reacting an intermediate of formula (VII), wherein W₂ represents a suitable leaving group, with an appropriate alcohol of formula HO—R_{5′} wherein R_{5′} represents C₁₋₆alkyl or hydroxyC₁₋₆alkyl in the presence of a suitable solvent 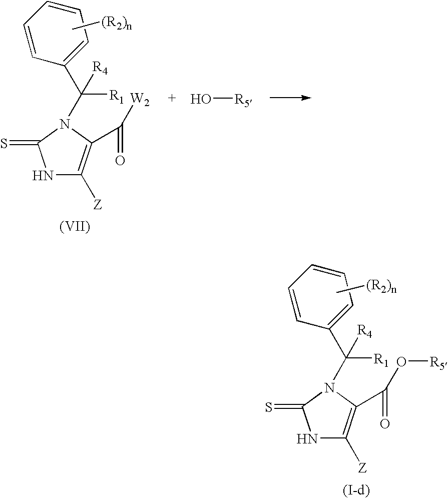
f) reacting an intermediate of formula (VII), wherein W₂ represents a suitable leaving group, with an intermediate of formula (VIII) in the presence of a suitable solvent 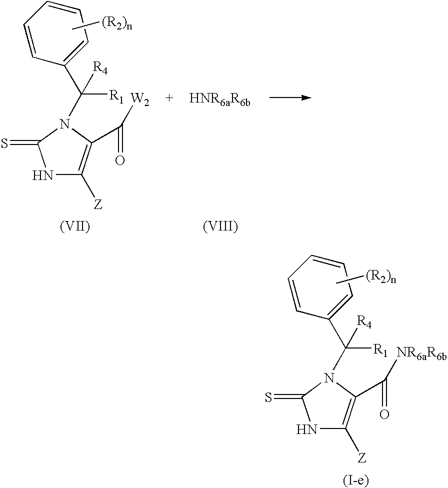
g) reacting an intermediate of formula (VII) with a suitable reducing agent in the presence of a suitable solvent 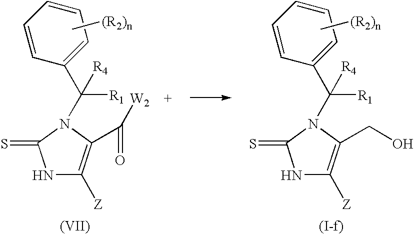
or, if desired, converting compounds of formula (I) into each other following art-known transformations, and further, if desired, converting the compounds of formula (I), into a therapeutically active non-toxic acid addition salt by treatment with an acid, or into a therapeutically active non-toxic base addition salt by treatment with a base, or conversely, converting the acid addition salt form into the free base by treatment with alkali, or converting the base addition salt into the free acid by treatment with acid; and, if desired, preparing stereochemically isomeric forms, quaternary amines or N-oxide forms thereof; wherein
R₁ represents hydrogen C₁₋₆ alkyl, C₃₋₇cycloalkyl, C₁₋₆alkyloxyC₁₋₆alkyl, di(C₁₋₆alkyl)aminoC₁₋₆alkyl, aryl, or heteroaryl;
each R₂ independently represents halo, C₁₋₆alkyl, C₁₋₆alkyloxy C₁₋₆alkylthio, polyhaloC₁₋₆alkyl, polyhaloC₁₋₆alkyloxy, cyano, aminocarbonyl amino mono- or di(C₁₋₄alkyl)amino, nitro, aryl or aryloxy;
R₃ represents hydrogen, cyano, C₁₋₆alkyl optionally substituted with hydroxy or C₁₋₆alkyloxy, C(═O)—O—R₅, C(═O)—NR₆ₐR_{6b}, C(═S)NR₆ₐR_{6b}, S(═O)₂—NR₆ₐR_{6b} or C(═O)—R₇;
R₄ represents hydrogen or C₁₋₆alkyl;
R₆ₐ and R_{6b} each independently represent hydrogen C₁₋₆alkyl amino mono- or di(C₁₋₄alkyl)amino, arylNH—, aminoC₁₋₆alkyl mono- or di(C₁₋₄alkyl)aminoC₁₋₆alkyl, C₁₋₆alkylcarbonylamino, aminocarbonylamino, C₁₋₆alkyloxy, carbonylamino or hydroxyC₁₋₆alkyl; or
R₆ₐ and R_{6b} taken together with the nitrogen to which they are attached form pyrrolidinyl imidazolidinyl, pyrazolidinyl, piperidinyl, piperazinyl, morpholinyl, thiomorpholinyl or piperazinyl substituted with C₁₋₆alkyl:
Z represents a cyclic ring system selected from 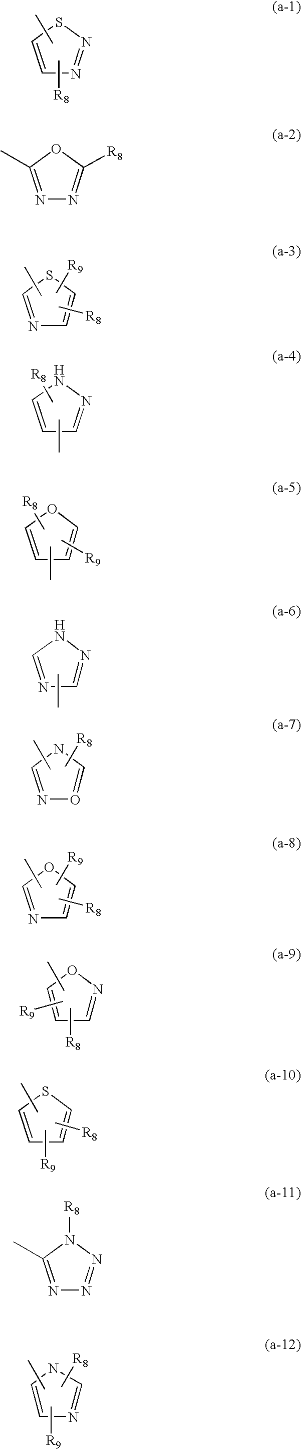 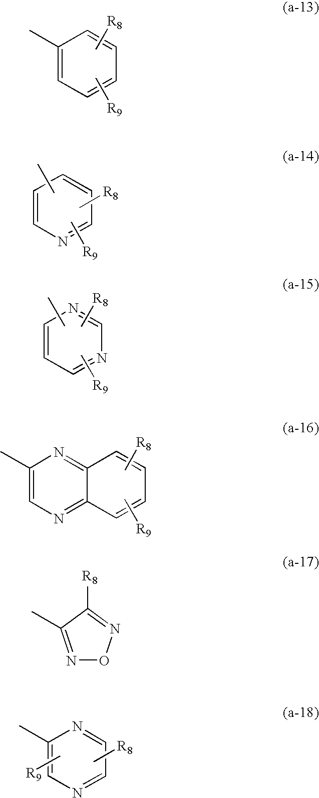
each R₈ independently represents hydrogen, halo, C₁₋₆alkyl, C₁₋₆alkyloxy, polyhaloC₁₋₆alkyl, polyhaloC₁₋₆alkyloxy, cyano, aminocarbonyl, mono- or di(C₁₋₄alkyl)aminocarbonyl amino, mono- or di(C₁₋₄alkyl)amino, hydroxyC₁₋₆alkylamino, aryl, aryloxy, piperidinyl, piperidinylamino, morpholinyl piperazinyl or nitro;
each R₉ independently represents hydrogen, halo or C₁₋₆alkyl;
n is 1, 2, 3, 4 or 5;
aryl represents phenyl or phenyl substituted with one, two, three, four or five substituents each independently selected from halo C₁₋₆alkyl, C₁₋₆alkyloxy polyhaloC₁₋₆alkyl, polyhaloC₁₋₆alkyloxy, cyano, aminocarbonyl mono- or di(C₁₋₄alkyl)aminocarbonyl, amino, mono- or di(C₁₋₄alkyl)amino, phenyloxy or nitro;
heteroaryl represents furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl pyrimidinyl, pyrazinyl, each of said heterocycles optionally being substituted with one or two substituents each independently selected from halo,
C₁₋₆alkyl, C₁₋₆alkyloxy, PolyhaloC₁₋₆alkyl, polyhaloC₁₋₆alkyloxy, cyano, aminocarbonyl mono- or di(C₁₋₄alkyl)aminocarbonyl, amino, mono- or di(C₁₋₄alkyl)amino or nitro.

37. A process of preparing a compound as defined in claim 22 comprising performing the reactions according to claim 36 starting from an intermediate wherein the carbon atom carrying the R₁ and R₄ substituent has the (S) configuration.

38. A compound of formula (IX-b-1-1) 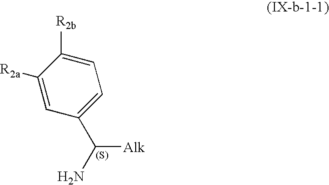
a N-oxide, a pharmaceutically acceptable addition salt or a quaternary amine thereof, wherein Alk represents methyl, ethyl or n-propyl, and each R₂ₐ and R_{2b} independently represents chloro, fluoro or trifluoromethyl provided that when R₂ₐ and R_{2b} are both chloro, then Alk is other than ethyl.

39. A compound according to claim 38 wherein the compound is [chemical structure]
